(19) United States
(12) Patent Application Publication
(10) Pub. No.: US 2025/0314963 A1
(43) Pub. Date: Oct. 9, 2025

(54) RESIST COMPOSITION AND PATTERN FORMING PROCESS

(71) Applicant: Shin-Etsu Chemical Co., Ltd., Tokyo (JP)

(72) Inventors: Satoshi Kusama, Joetsu-shi (JP); Kousuke Ohyama, Joetsu-shi (JP); Shun Kikuchi, Joetsu-shi (JP); Masaki Ohashi, Joetsu-shi (JP)

(73) Assignee: Shin-Etsu Chemical Co., Ltd., Tokyo (JP)

(21) Appl. No.: 19/089,370

(22) Filed: Mar. 25, 2025

(30) Foreign Application Priority Data
Apr. 3, 2024 (JP) 2024-059980

(51) Int. Cl. G03F 7/004; C07C 69/63; G03F 7/00; G03F 7/038; G03F 7/039

(52) U.S. Cl. CPC G03F 7/0045; C07C 69/63; G03F 7/0046; G03F 7/0382; G03F 7/0392; G03F 7/70033

(57) ABSTRACT

The resist composition comprises a hypervalent iodine compound having the formula (1), a carboxy group-containing polymer, and a solvent. The resist composition above exhibits a high sensitivity and resolution when processed by photolithography using high-energy radiation, typically EB and EUV lithography, and a pattern forming process using the resist composition.

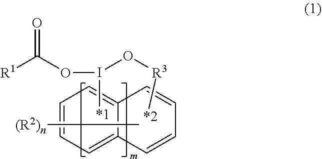

### CROSS-REFERENCE TO RELATED APPLICATIONS

This non-provisional application claims priority under 35 U.S.C. § 119(a) on Patent Application No. 2024-059980 filed in Japan on Apr. 3, 2024, the entire contents of which are hereby incorporated by reference.

### TECHNICAL FIELD

This invention relates to a resist composition and a patterning process.

### BACKGROUND ART

While a higher integration density, higher operating speed and lower power consumption of LSIs are demanded to comply with the expanding IoT market, the effort to reduce the pattern rule is in rapid progress. The wide-spreading logic device market drives forward the miniaturization technology. As the advanced miniaturization technology, microelectronic devices of 10-nm node are manufactured in a mass scale by the double, triple or quadro-patterning version of the immersion ArF lithography. Active research efforts have been made on the manufacture of 7-nm node devices by the next generation EUV lithography of wavelength 13.5 nm.

As the feature size is reduced, image blurs due to acid diffusion become a problem (see Non-Patent Document 1). To insure resolution for fine patterns with a processing dimension of 45 nm et seq., not only an improvement in dissolution contrast is requisite, but the control of acid diffusion is also important (see Non-Patent Document 2). Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

Addition of an acid generator capable of generating a bulky acid is effective for suppressing acid diffusion. Therefore, it has been proposed to copolymerize a polymer with an acid generator in the form of an onium salt having a polymerizable unsaturated bond. With respect to the patterning of a resist film to a processing dimension of 16 nm et seq., it is believed impossible in the light of acid diffusion to form such a pattern from a chemically amplified resist composition. It would be desirable to have a non-chemically amplified resist composition.

A typical non-chemically amplified resist composition material is polymethyl methacrylate (PMMA). PMMA is a positive resist material which increases solubility in organic solvent developer through the mechanism that the molecular weight becomes lower as a result of scission of the main chain upon EUV exposure.

Hydrogensilsesquioxane (HSQ) is a negative resist material which turns insoluble in alkaline developer through crosslinking by condensation reaction of silanol generated upon EUV exposure. Also, chlorine-substituted calixarene functions as negative resist material. Since these negative resist materials have a small molecular size prior to crosslinking and avoid any blur caused by acid diffusion, they exhibit reduced edge roughness and very high resolution. They are thus used as a pattern transfer material for representing the resolution limit of the exposure tool. However, these materials are insufficient in sensitivity, with further improvements being needed.

One of the causes that retard the development of EUV lithography materials is a small number of photons available with EUV exposure. The energy of EUV is extremely higher than that of ArF excimer laser. The number of photons available with EUV exposure is 1/14 of the number by ArF exposure. The size of pattern features formed by the EUV lithography is less than half the size by the ArF lithography. Therefore, the EUV lithography is quite sensitive to a variation of photon number. A variation in number of photons in the radiation region of extremely short wavelength is shot noise as a physical phenomenon. It is impossible to eliminate the influence of shot noise. Attention is thus paid to stochastics. While it is impossible to eliminate the influence of shot noise, discussions are held how to reduce the influence. There is observed a phenomenon that under the influence of shot noise, values of CDU and LWR are increased and holes are blocked at a probability of one several millionth. The blockage of holes leads to electric conduction failure to prevent transistors from operation, adversely affecting the performance of an overall device. In view of practically acceptable sensitivity, resist compositions based on PMMA or HSQ are largely affected by stochastics, failing to gain the desired resolution.

As the means for reducing the influence of shot noise on the resist side, it is noteworthy to incorporate an element having high EUV absorption. Patent Document 1 discloses a chemically amplified resist composition containing highly EUV-absorbing iodine atoms. However, as mentioned above, the chemically amplified resist composition cannot reach the resolution desired in the EUV lithography where processing dimensions become smaller than ever.

Patent Document 2 discloses a negative resist composition comprising a tin compound. Based on tin element having high EUV absorption, this resist composition is improved in stochastics and achieves a high sensitivity and high resolution. Such so called metal resist compositions, however, suffer from many problems including low solubility in resist solvents, poor shelf stability, and defectiveness due to post-etching residues.

To address this, Patent Document 3 discloses a positive resist composition comprising a hypervalent iodine compound. Based on iodine element having high EUV absorption, this resist composition is improved in stochastics and achieves a high sensitivity and high resolution like metal resists. Further, since such positive resist compositions are composed only of organic molecules, they can avoid the problems of metal resists which are poor solubility in a developer and defects due to residues. However, performance as a resist material is not satisfactory, and development useful for formation of finer patterns is desired.

### CITATION LIST

Patent Document 1: JP-A 2018-5224
Patent Document 2: JP-W 2021-503482
Patent Document 3: JP-A 2023-167368
Non-Patent Document 1: SPIE Vol. 5039 p1 (2003)
Non-Patent Document 2: SPIE Vol. 6520 p65203L-1 (2007)

### SUMMARY OF THE INVENTION

An object of the invention is to provide a resist composition which exhibits a high sensitivity and resolution when processed by photolithography using high-energy radiation, typically EB and EUV lithography, and a pattern forming process using the resist composition.

The inventors have found that a resist composition based on a hypervalent iodine compound having a predetermined carboxylate ligand and a carboxy group-containing polymer has a very high sensitivity, forms a resist film having a satisfactory resolution, and is thus quite useful in precise micropatterning.

The invention provides a resist composition and a pattern forming process described below.

1. A resist composition comprising a hypervalent iodine compound having the formula (1), a carboxy group-containing polymer, and a solvent:

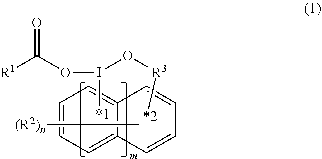

wherein m is 0 or 1, n is an integer of 0 to 4 when m is 0, and an integer of 0 to 6 when m is 1,
R¹ is halogen, or a C₁-C₁₀ hydrocarbyl group which may contain a heteroatom,
R² is halogen, or a C₁-C₄₀ hydrocarbyl group which may contain a heteroatom, R² may be the same or different when n is 2 or more, a plurality of R² may bond together to form a ring with the aromatic ring carbon atoms to which they are attached,
R³ is a carbonyl group, or C₁-C₁₀ hydrocarbylene group which may contain a heteroatom, and
*1 and *2 each designate a valence bond to a carbon atom in an aromatic ring, provided that *1 and *2 bond to adjacent carbon atoms in the aromatic ring.

2. The resist composition of the item 1 wherein the carboxy group-containing polymer contains a recurring unit having the formula (2):

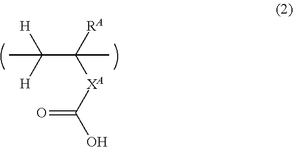

wherein R^{A} is a hydrogen atom, halogen, methyl group, or trifluoromethyl group,
X^{A} is a single bond, phenylene group, naphthylene group, or *—C(═O)—O—X^{A1}— wherein X^{A1} is a C₁-C₁₀ saturated hydrocarbylene group, phenylene group, or naphthylene group, the saturated hydrocarbylene group may contain at least one selected from a hydroxy group, an ether bond, an ester bond and a lactone ring, and * designates a valence bond to a carbon atom in a main chain.

3. The resist composition of the item 1 or 2, further comprising a hypervalent iodine compound having the formula (3):

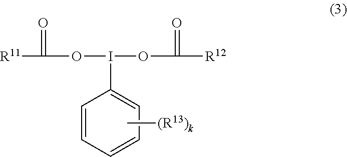

wherein k is an integer of 0 to 5,
R¹¹ and R¹² are each independently halogen or a C₁-C₁₀ hydrocarbyl group which may contain a heteroatom, R¹¹ and R¹² may bond together to form a ring with the carbon atoms to which they are attached, and atoms between the carbon atoms.
R¹³ is halogen, or a C₁-C₄₀ hydrocarbyl group which may contain a heteroatom, R¹³ may be the same or different when k is 2, 3, 4 or 5, and a plurality of R¹³ may bond together to form a ring with the aromatic ring carbon atoms to which they are attached.

4. A laminate comprising a substrate, and a resist film obtained from the resist composition of any one of the items 1 to 3.

5. The laminate of the item 4 comprising a resist underlayer film between the substrate and the resist film.

6. A pattern forming process comprising the steps of applying the resist composition of any one of the items 1 to 3 onto a substrate, or an underlayer film laminated on a substrate to form a resist film thereon, exposing the resist film to a high-energy radiation, and developing the exposed resist film in a developer.

7. The pattern forming process of the item 6, wherein the high-energy radiation is an electron beam or an extreme ultraviolet radiation.

8. The pattern forming process of the item 6 or 7, wherein the developer dissolves exposed regions and does not dissolve unexposed regions.

9. The pattern forming process of the item 6 or 7, wherein the developer dissolves unexposed regions and does not dissolve exposed regions.

10. A laminate comprising a substrate, and a resist film on the substrate, wherein the resist film contains a polymer containing a recurring unit having the formula (4).

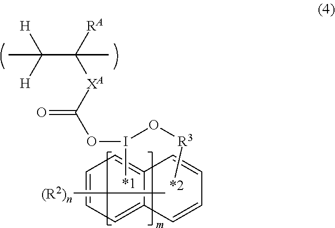

wherein m is 0 or 1, n is an integer of 0 to 4 when m is 0, and an integer of 0 to 6 when m is 1,
R² is halogen, or a C₁-C₄₀ hydrocarbyl group which may contain a heteroatom, R² may be the same or different when n is 2 or more, a plurality of R² may bond together to form a ring with the aromatic ring carbon atoms to which they are attached,
R³ is a carbonyl group, or C₁-C₁₀ hydrocarbylene group which may contain a heteroatom, and
*1 and *2 each designate a valence bond to a carbon atom in an aromatic ring, Note that *1 and *2 bond to adjacent carbon atoms in the aromatic ring.
R^{A} is a hydrogen atom, halogen, methyl group, or trifluoromethyl group,
X^{A} is a single bond, phenylene group, naphthylene group, or *—C(═O)—O—X^{A1}— wherein X^{A1} is a C₁-C₁₀ saturated hydrocarbylene group, phenylene group, or naphthylene group, the saturated hydrocarbylene group may contain at least one selected from a hydroxy group, an ether bond, an ester bond and a lactone ring, and * designates a valence bond to a carbon atom in a main chain.

11. The laminate of the item 10 comprising a resist underlayer film between the substrate and the resist film.

12. A pattern forming process comprising the steps of exposing the resist film of the laminate of the item 10 or 11 to high-energy radiation, and developing the exposed resist film in a developer.

13. The pattern forming process of the item 12, wherein the high-energy radiation is an electron beam or an extreme ultraviolet radiation.

14. The pattern forming process of the item 12 or 13, wherein the developer dissolves exposed regions and does not dissolve unexposed regions.

15. The pattern forming process of the item 12 or 13, wherein the developer dissolves unexposed regions and does not dissolve exposed regions.

### Advantageous Effects of the Invention

The resist composition exhibits both high sensitivity and resolution when processed by EB and EUV lithography and is quite useful in micropatterning.

### DETAILED DESCRIPTION OF THE INVENTION

### [Resist Composition]

One embodiment of the invention is a resist composition comprising a hypervalent iodine compound having a predetermined carboxylate ligand, a carboxy group-containing polymer, and a solvent.

### [Hypervalent Iodine Compound]

The hypervalent iodine compound is a three-coordinate hypervalent iodine compound having the formula (1).

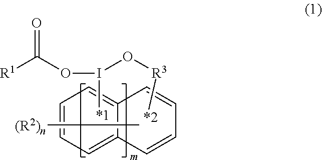

In the formula (1), m is an integer of 0 or 1. n is an integer of 0 to 4 when m is 0, and an integer of 0 to 6 when m is 1. n is preferably 0, 1, 2, 3 or 4, more preferably 0, 1, 2 or 3, still more preferably 0, 1 or 2, most preferably 0 or 1.

In formula (1), R¹ is halogen, or a C₁-C₁₀ hydrocarbyl group which may contain a heteroatom. Examples of the halogen include fluorine, chlorine, bromine and iodine atoms. The C₁-C₁₀ hydrocarbyl group may be saturated or unsaturated and straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₁₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decanyl and adamantyl groups; C₂-C₁₀ alkenyl groups such as vinyl and allyl groups; C₆-C₁₀ aryl groups such as phenyl and naphthyl groups; and combinations thereof. Also included are hydrocarbyl groups in which some or all of the hydrogen atoms are substituted by a moiety containing a heteroatom such as oxygen, sulfur, nitrogen, or halogen, and some constituent —CH₂— is replaced by a moiety containing a heteroatom such as oxygen, sulfur, or nitrogen, so that the group may contain hydroxy, cyano, halogen, carbonyl, ether bond, thioether bond, ester bond, sulfonic ester bond, carbonate bond, carbamate bond, lactone ring, sultone ring, or carboxylic anhydride (—C(═O)—O—C(═O)—). R¹ is preferably a C₁-C₄ hydrocarbyl or C₁-C₄ fluorinated hydrocarbyl group, more preferably a C₁-C₄ hydrocarbyl group.

In formula (1), R² is halogen, or a C₁-C₄₀ hydrocarbyl group which may contain a heteroatom. Examples of the halogen include fluorine, chlorine, bromine and iodine atoms. The C₁-C₄₀ hydrocarbyl group may be saturated or unsaturated and straight, branched or cyclic. Examples thereof include C₁-C₄₀ alkyls such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decanyl, adamantyl and adamantylmethyl groups; C₆-C₄₀ aryl groups such as phenyl, naphthyl and anthracenyl groups; and combinations thereof. Also included are hydrocarbyl groups in which some or all of the hydrogen atoms are substituted by a moiety containing a heteroatom such as oxygen, sulfur, nitrogen, or halogen, and some constituent —CH₂— is replaced by a moiety containing a heteroatom such as oxygen, sulfur, or nitrogen, so that the group may contain hydroxy, cyano, halogen, carbonyl, ether bond, thioether bond, ester bond, sulfonic ester bond, carbonate bond, carbamate bond, lactone ring, sultone ring, or carboxylic anhydride (—C(═O)—O—C(═O)—). R² may be the same or different when n is 2 or more. A plurality of R² may bond together to form a ring with the aromatic ring carbon atoms to which they are attached.

In the formula (1), R³ is a carbonyl group, or C₁-C₁₀ hydrocarbylene group which may contain a heteroatom. The C₁-C₁₀ hydrocarbylene group may be saturated or unsaturated and straight, branched or cyclic. Examples thereof include C₁-C₁₀ alkanediyl groups such as methanediyl, ethane-1,1-diyl, ethane-1,2-diyl, propane-1,1-diyl, propane-1,2-diyl, propane-1,3-diyl, propane-2,2-diyl, butane-2,3-diyl, butane-1,4-diyl, 2-methylpropane-1,2-diyl, pentane-1,5-diyl, hexane-1,6-diyl, heptane-1,7-diyl, octane-1,8-diyl, nonane-1,9-diyl, and decane-1,10-diyl; C₃-C₁₀ cyclic saturated hydrocarbylene groups such as cyclopentanediyl, cyclohexanediyl, norbornanediyl, adamantanediyl and tricyclo[5.2.1.0^{2.6}]decanediyl groups; C₂-C₁₀ alkenediyl groups such as vinylene and propenylene groups; C₆-C₁₀ arylene groups such as phenylene, methylphenylene, ethylphenylene, n-propylphenylene, isopropylphenylene, n-butylphenylene and naphthylene groups; and combinations thereof. Also included are hydrocarbylene groups in which some or all of the hydrogen atoms are substituted by a moiety containing a heteroatom such as oxygen, sulfur, nitrogen, or halogen, and some constituent —CH₂— is replaced by a moiety containing a heteroatom such as oxygen, sulfur, or nitrogen, so that the group may contain hydroxy, cyano, alkyl halide, halogen, carbonyl, ether bond, thioether bond, ester bond, sulfonic ester bond, carbonate bond, carbamate bond, lactone ring, sultone ring, or carboxylic anhydride (—C(═O)—O—C(═O)—). R³ is preferably a carbonyl, C₁-C₄ hydrocarbylene or C₁-C₄ fluorinated hydrocarbylene group.

In the formula (1), *1 and *2 each designate a valence bond to a carbon atom in an aromatic ring. Note that *1 and *2 bond to adjacent carbon atoms in the aromatic ring. There may be the following four types of the combination of *1, *2 and m:

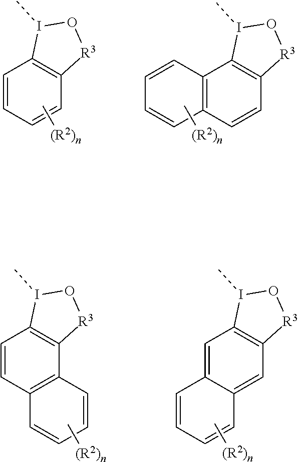

wherein n, R² and R³ are as defined above, and the broken line designates valent bond to R¹—C(═O)—O—.

Examples of the hypervalent iodine compound having the formula (1) are shown below, but not limited thereto. In the following formula, Me is a methyl group.

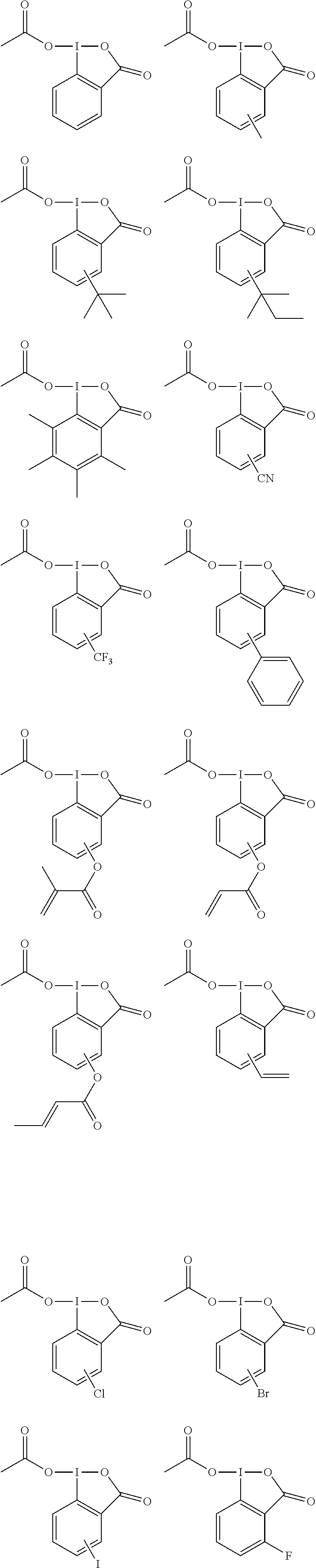

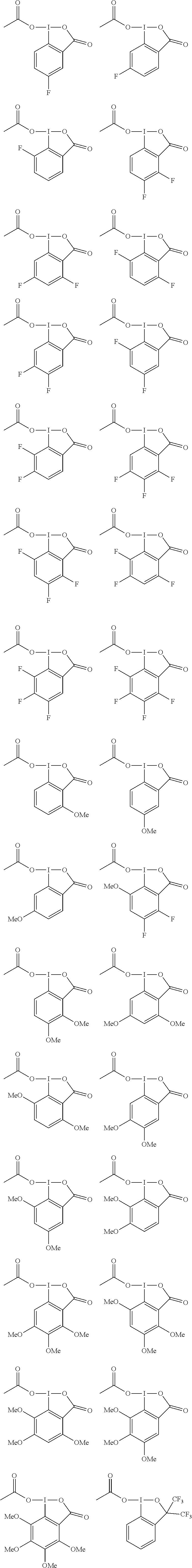

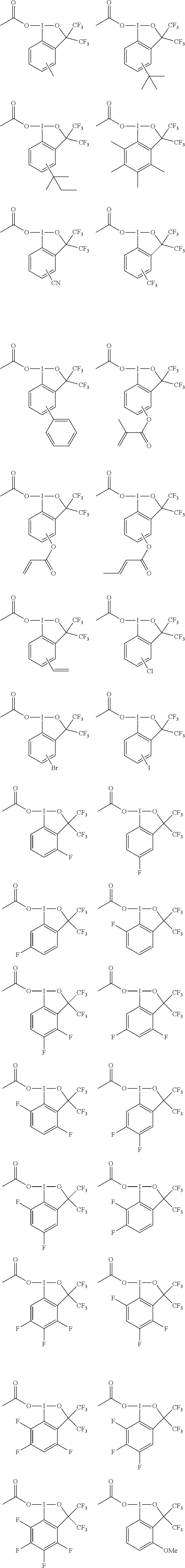

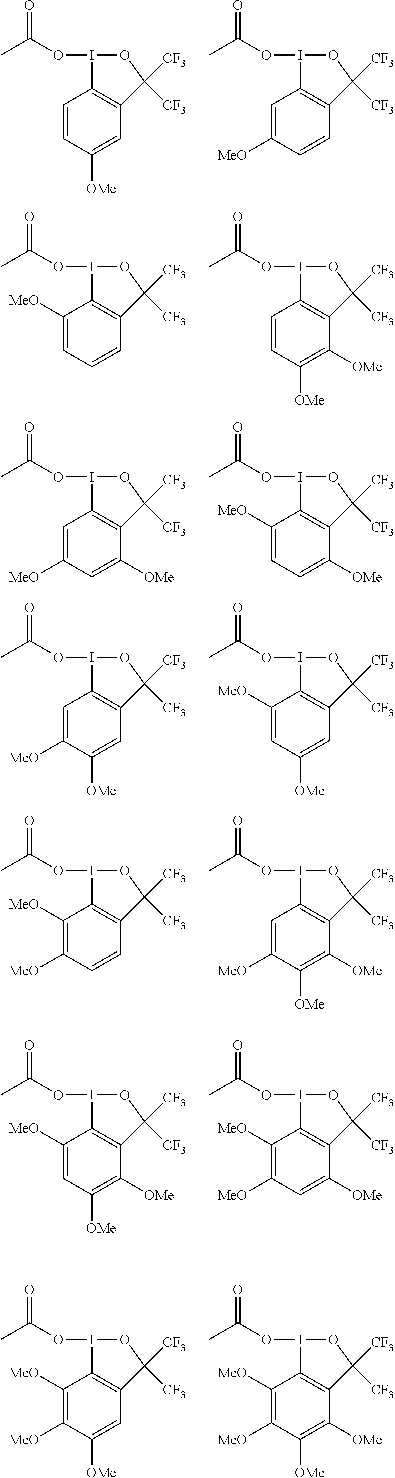

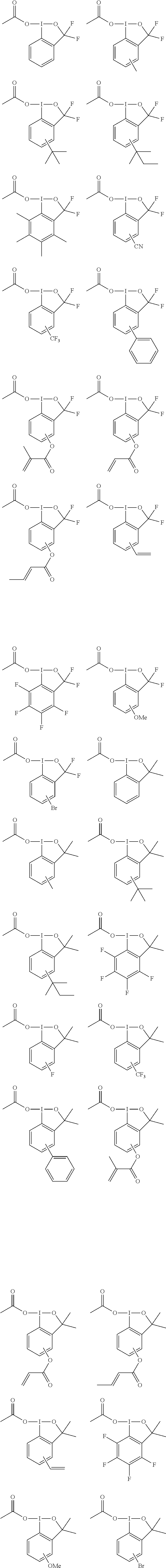

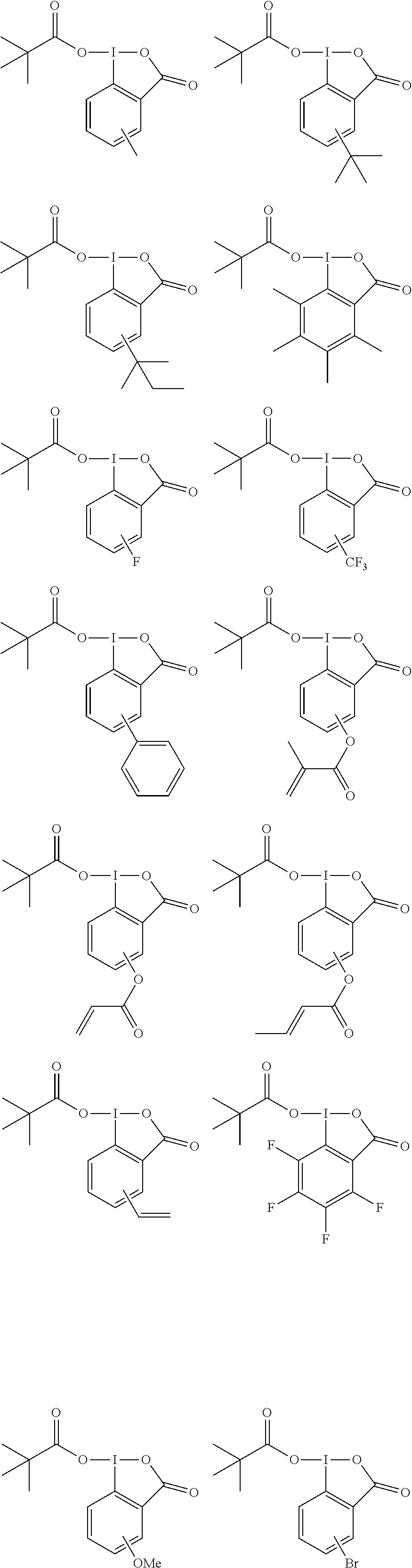

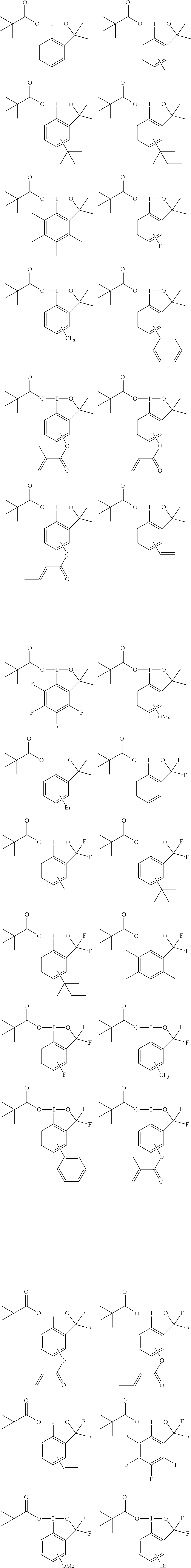

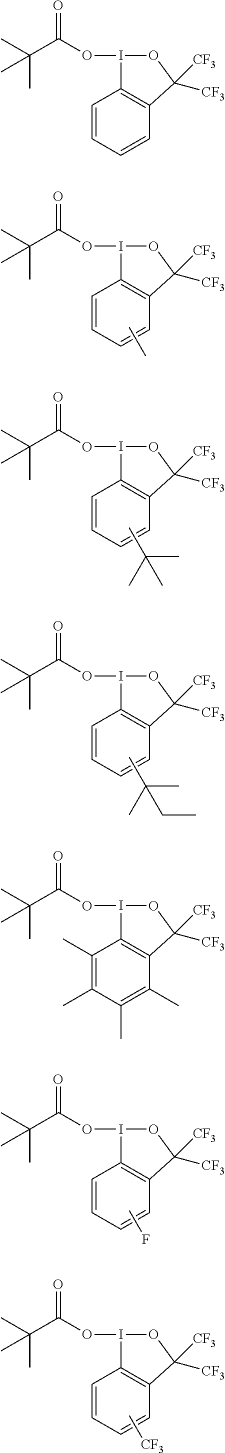

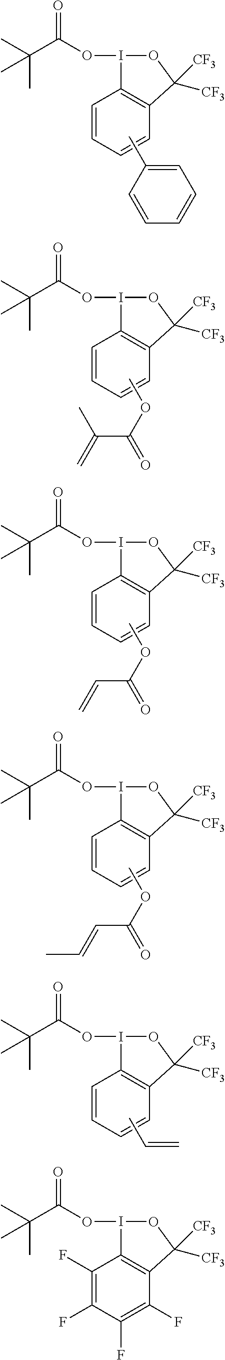

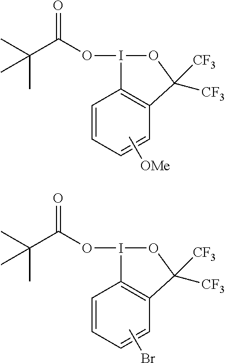

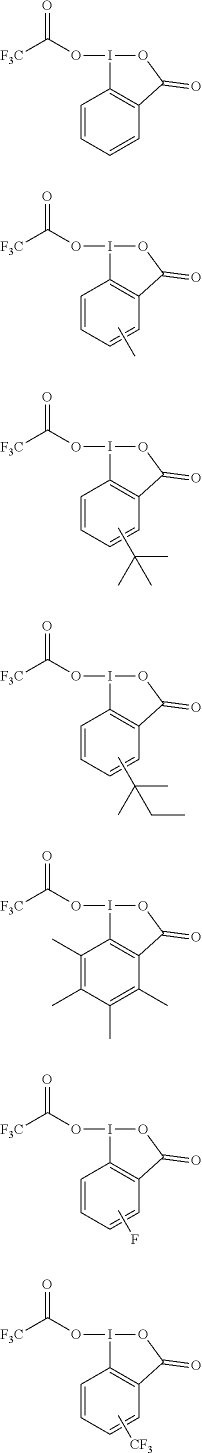

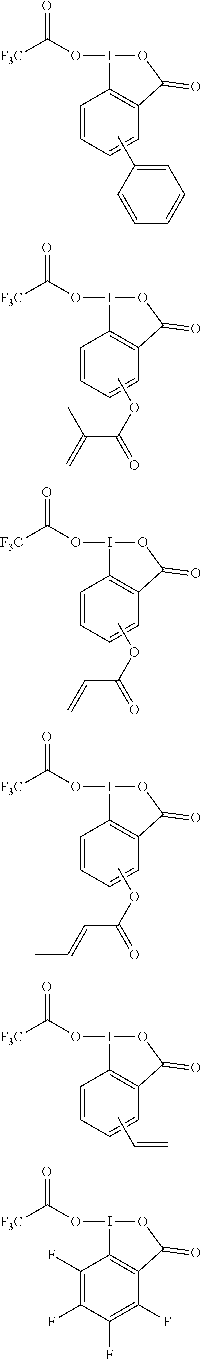

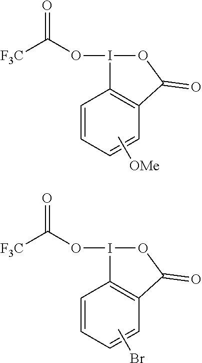

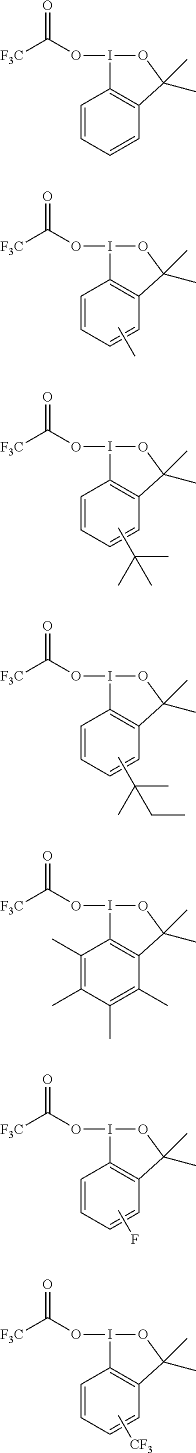

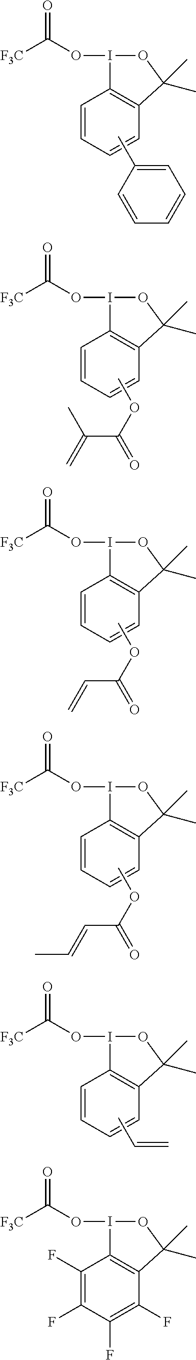

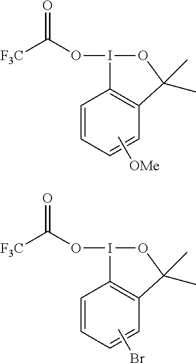

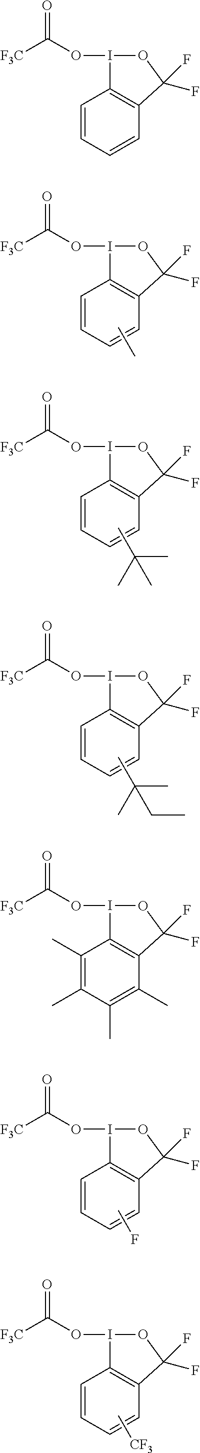

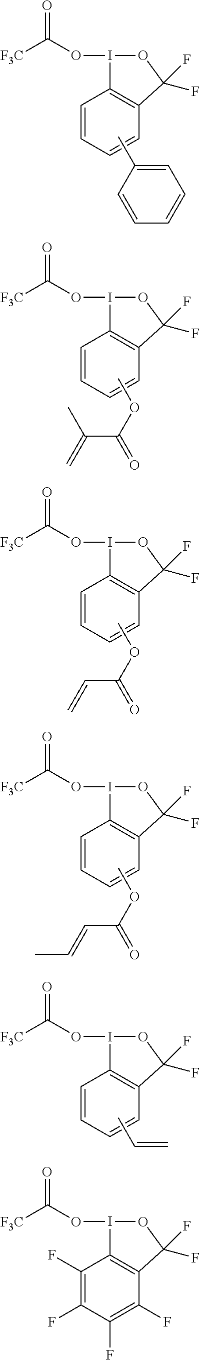

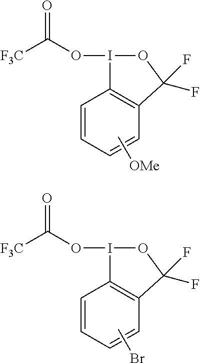

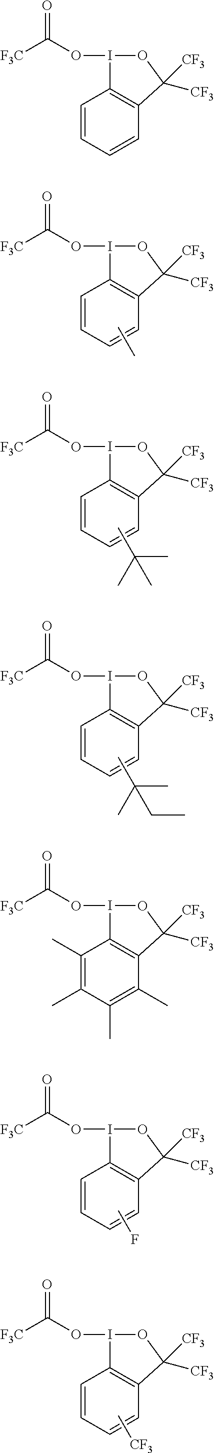

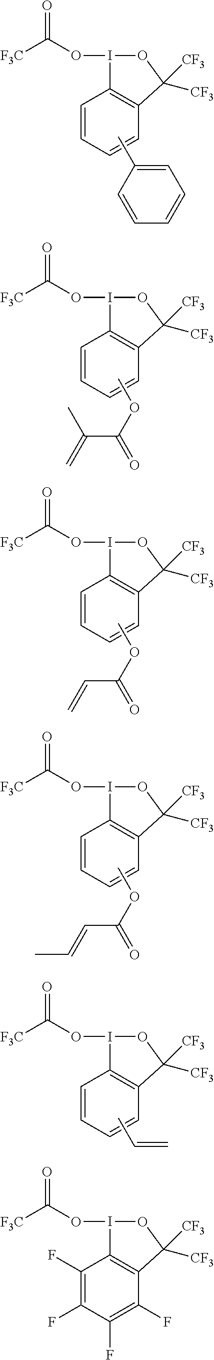

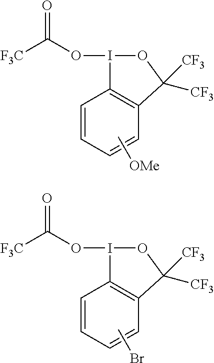

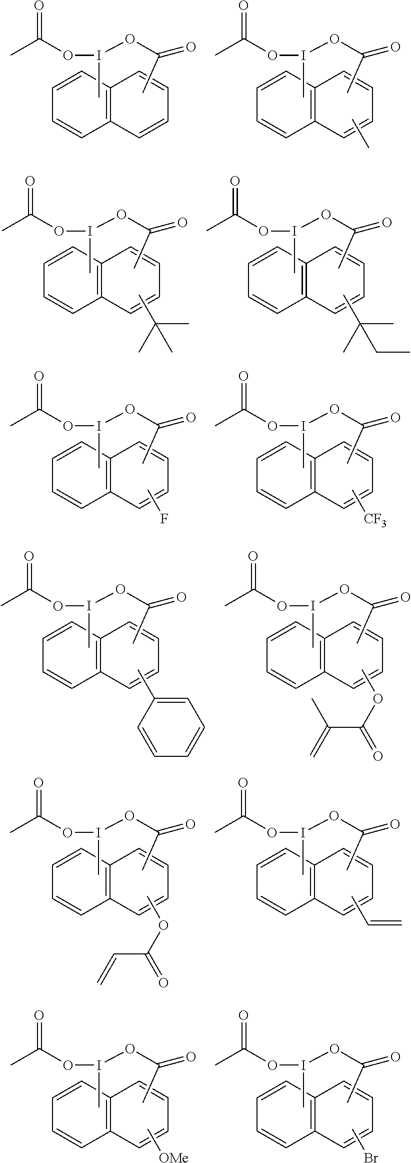

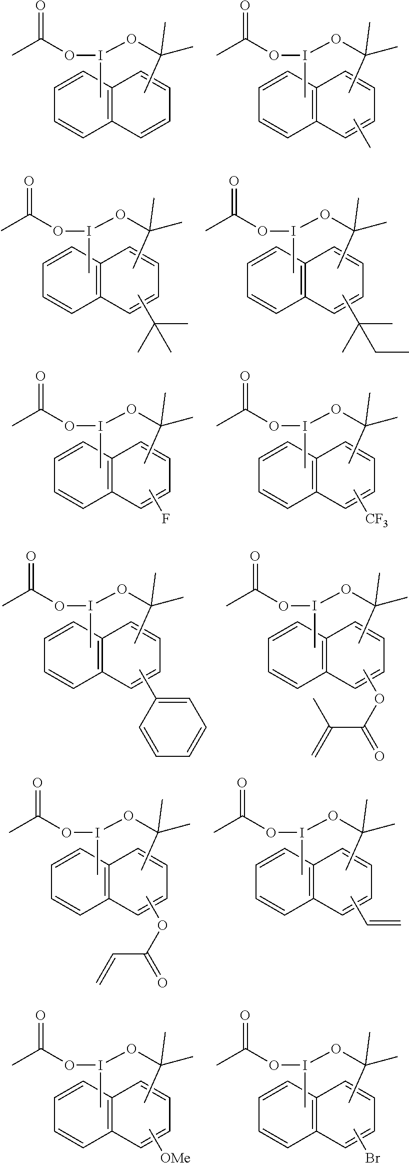

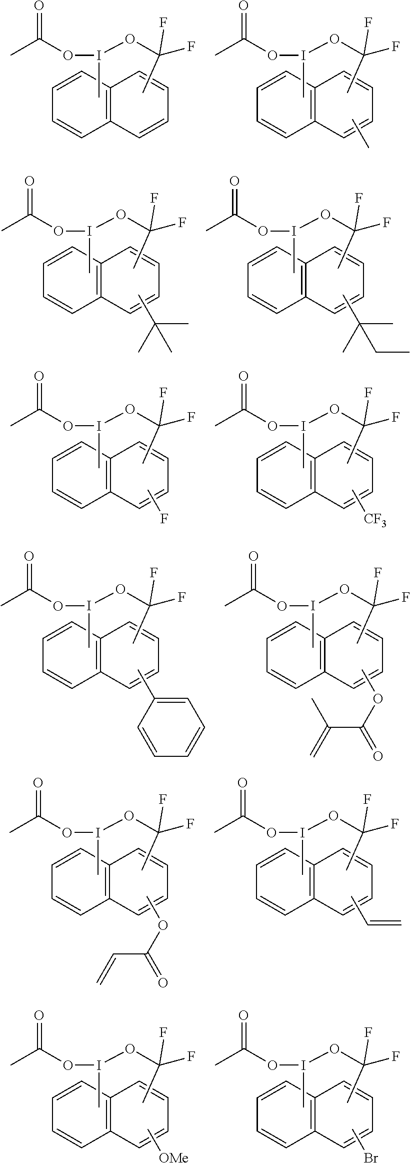

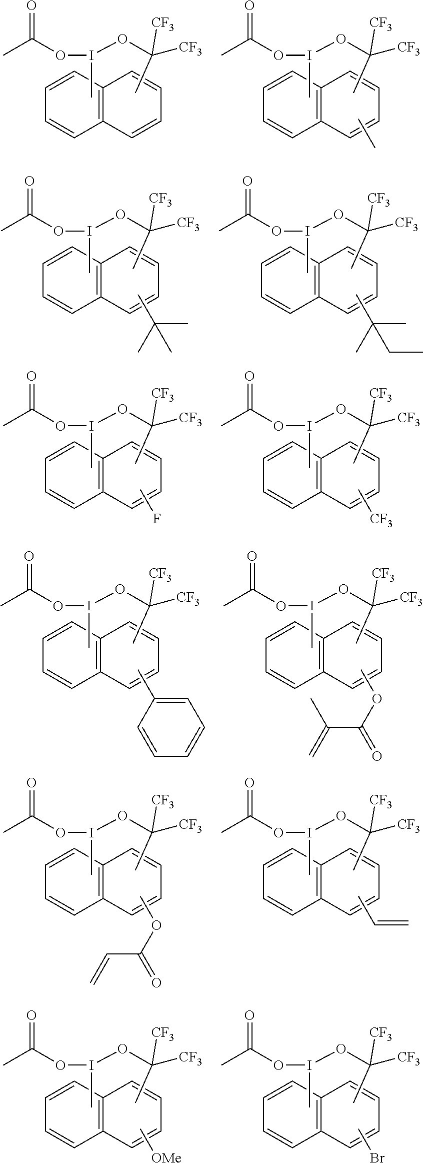

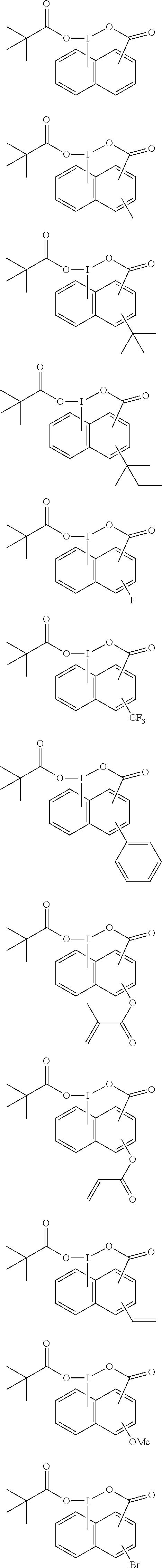

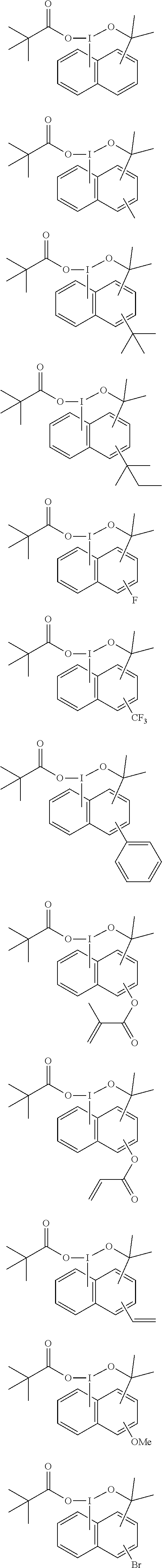

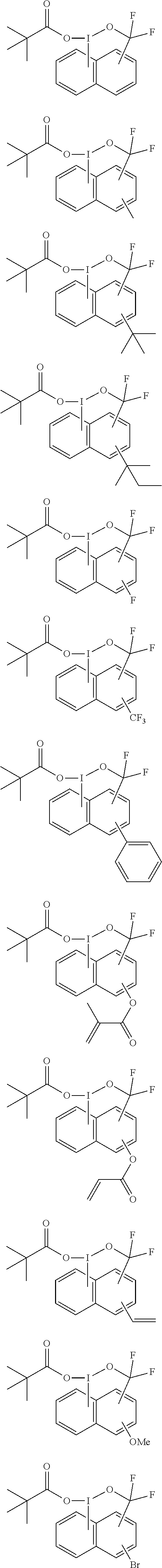

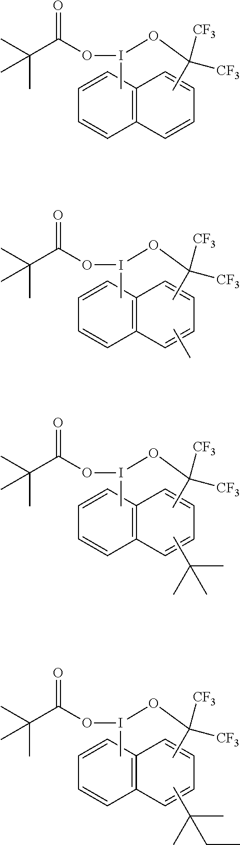

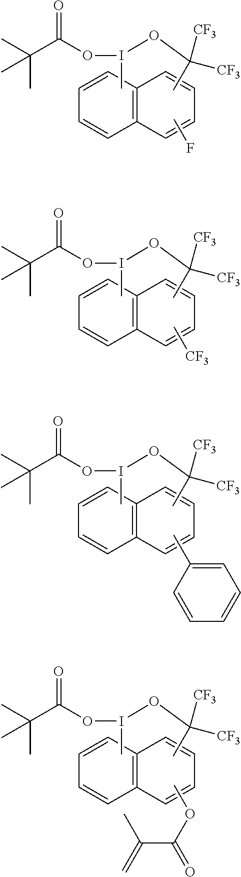

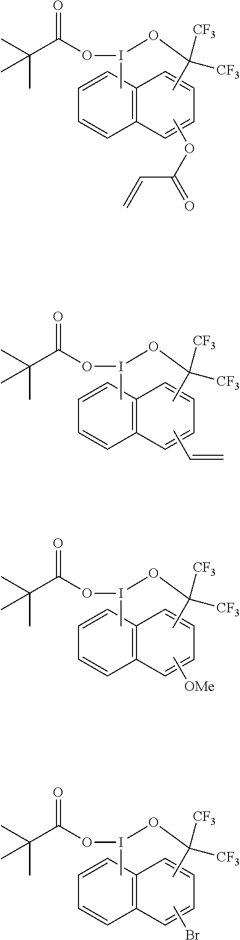

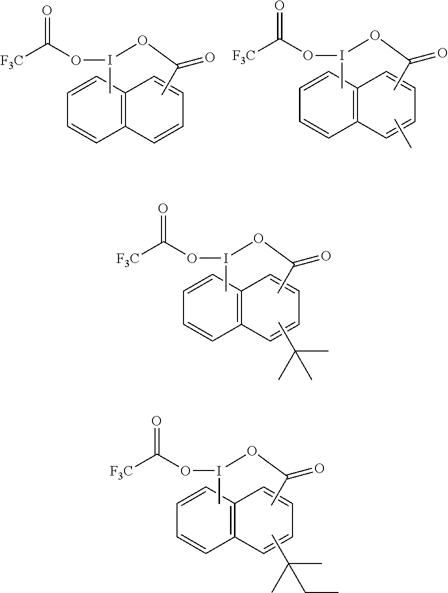

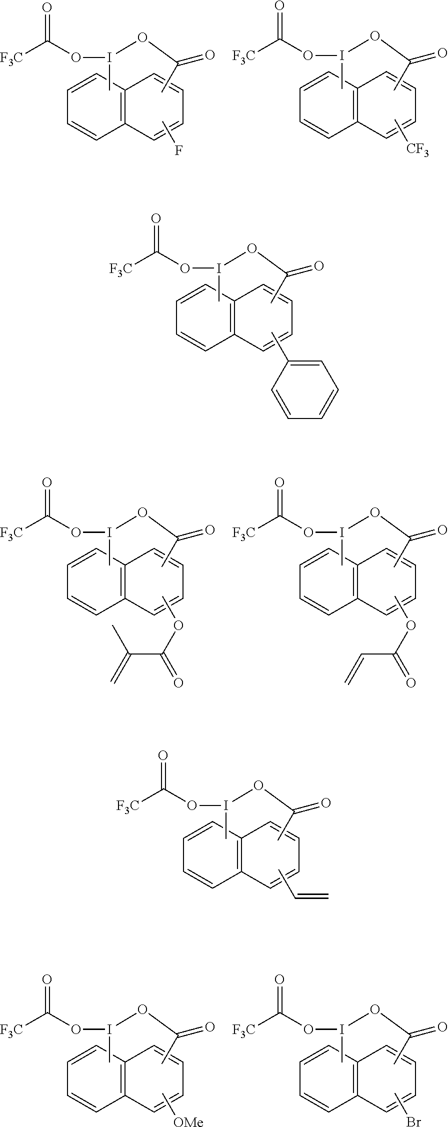

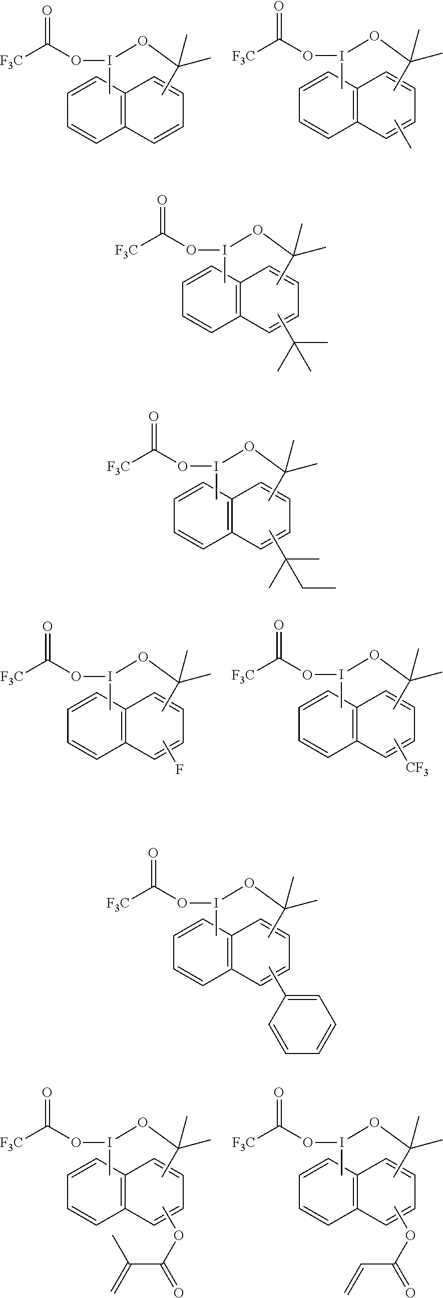

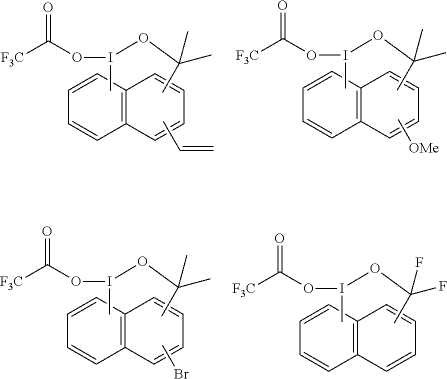

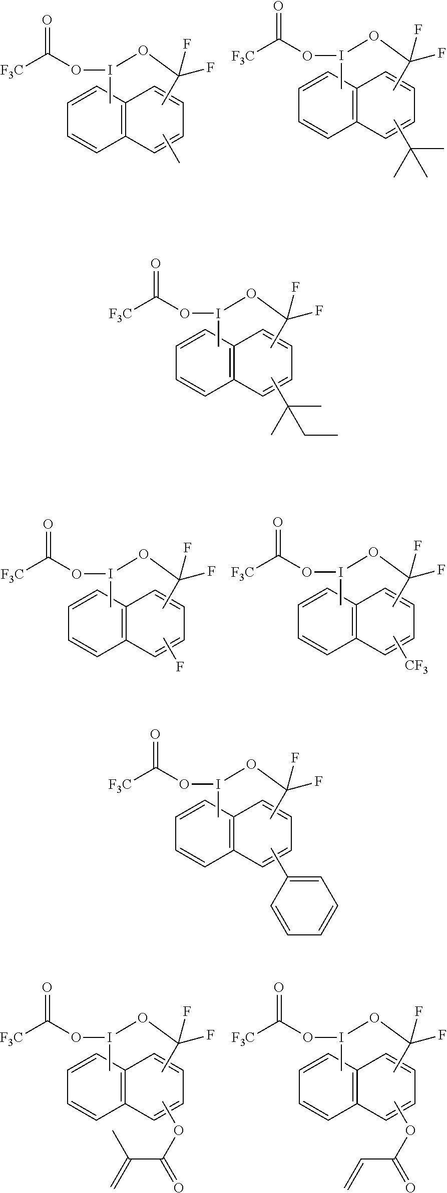

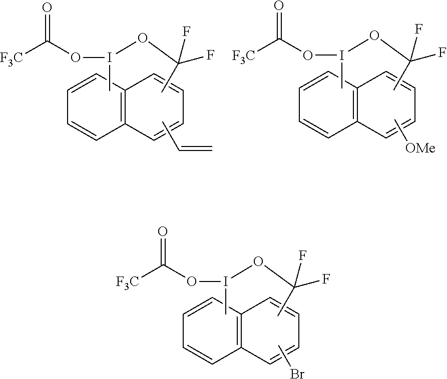

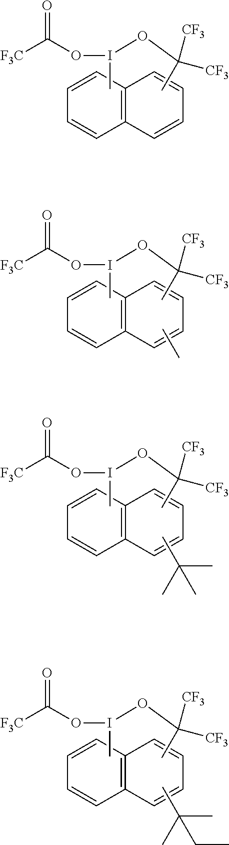

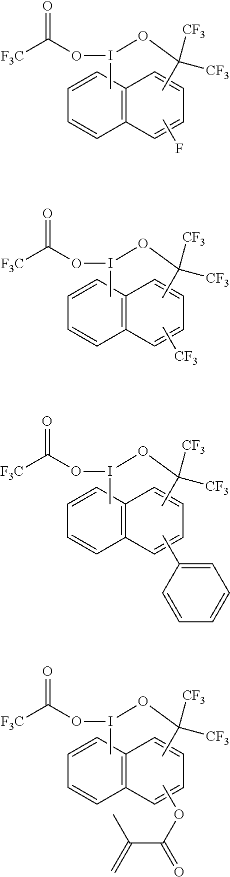

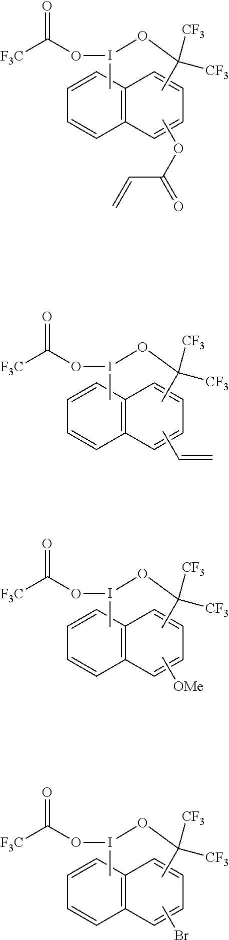

### [Carboxy Group-Containing Polymer]

The carboxy group-containing polymer preferably contains a carboxy group containing recurring unit. Preferably, the carboxy group-containing recurring unit has the formula (2).

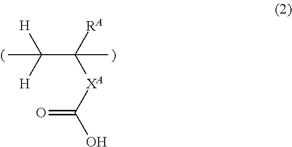

In the formula (2), R^{A} is a hydrogen atom, halogen, methyl group, or trifluoromethyl group. X^{A} is a single bond, phenylene group, naphthylene group, or *—C(═O)—O—X^{A1}. X^{A1} is a C₁-C₁₀ saturated hydrocarbylene group, phenylene group, or naphthylene group, the saturated hydrocarbylene group may contain at least one selected from a hydroxy group, an ether bond, an ester bond and a lactone ring. * designates a valence bond to a carbon atom in a main chain.

Examples of the carboxy group-containing recurring unit are shown below, but not limited thereto. Herein, R^{A} is as defined above.

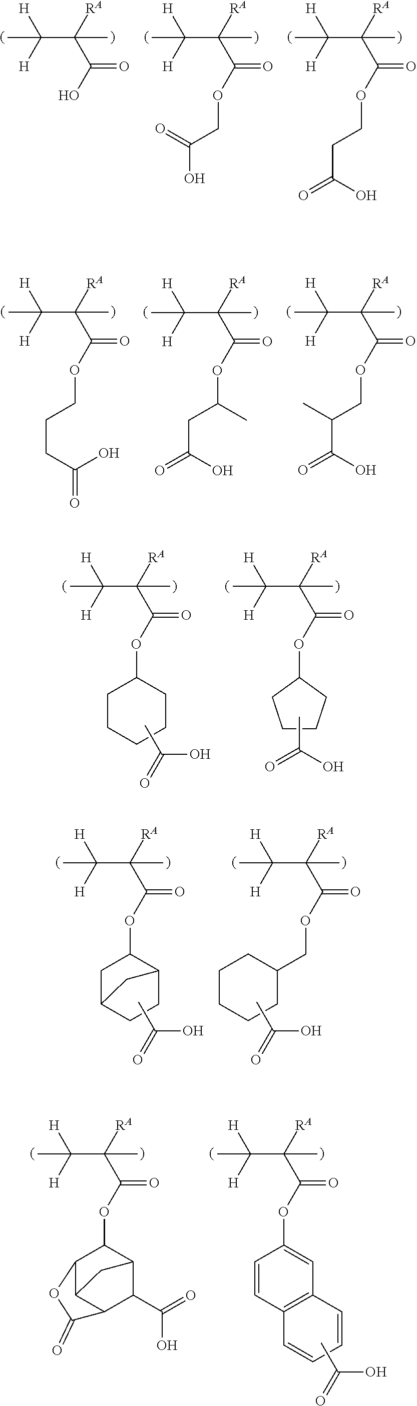

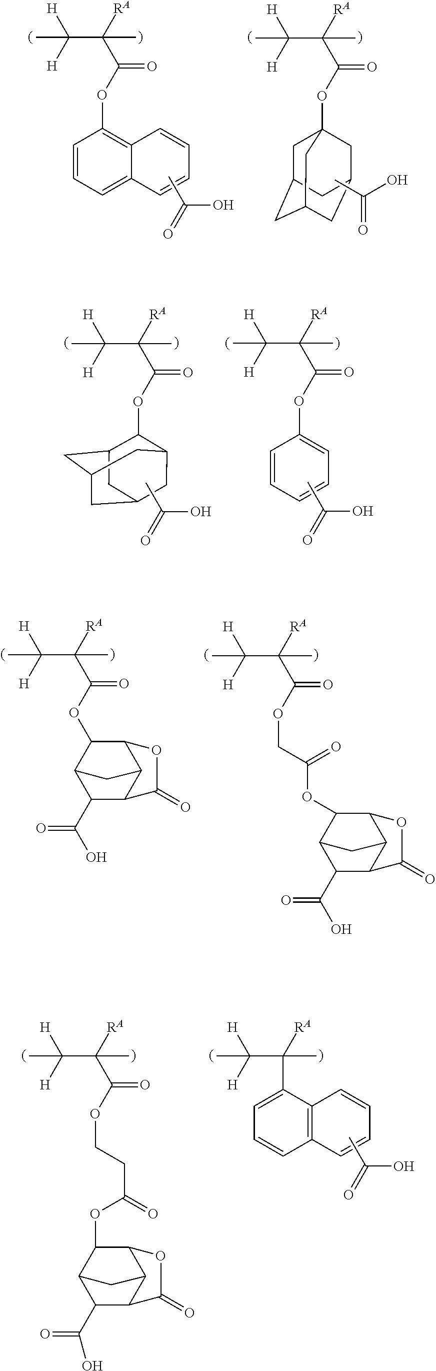

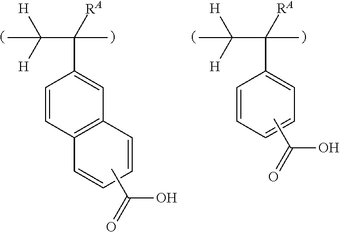

The carboxy group-containing polymer may further contain a recurring unit other than the carboxy group-containing recurring unit (hereinafter, also referred to as another recurring unit). Another recurring unit is not particularly limited, and a recurring unit is preferable that is capable of improving the solubility, in a solvent, of an insoluble polymer containing only a recurring unit having a carboxy group. Another recurring unit preferably has a C₁-C₂₀ hydrocarbyl group which may contain at least one selected from halogen, a hydroxy group, a cyano group, a carbonyl group, a nitro group, a sulfone group, an amino group, an isocyanate group, an amide group, an imide group, an ester bond, an ether bond, a sulfide bond, a carbonate bond, a lactone ring, and a sultone ring.

Examples of another recurring unit are shown below, but not limited thereto. Herein R^{A} is as defined above, and X^{B} are each independently —CH₂— or —O—.

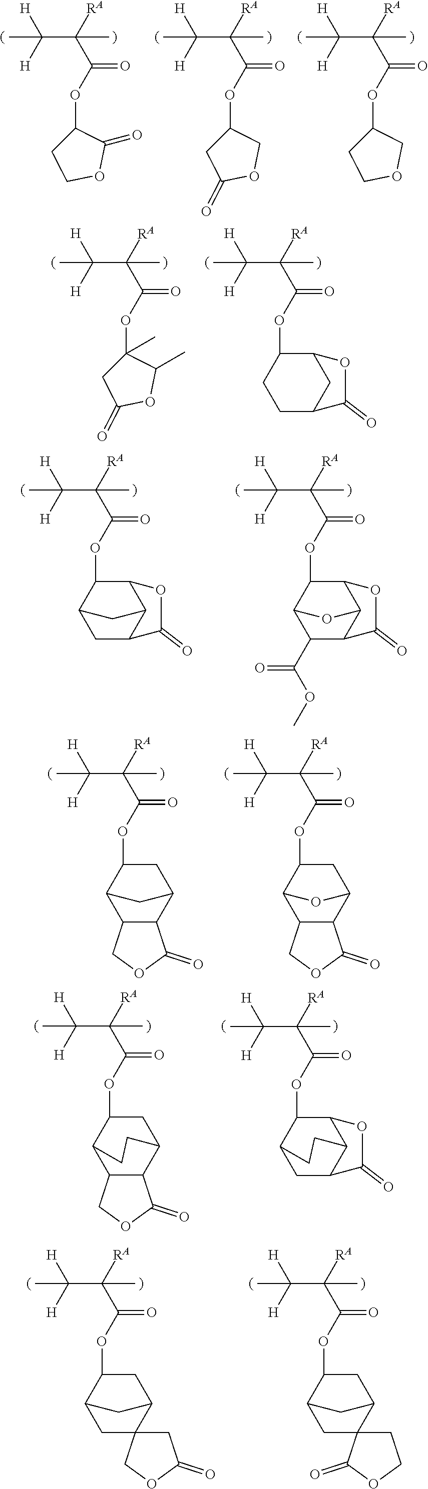

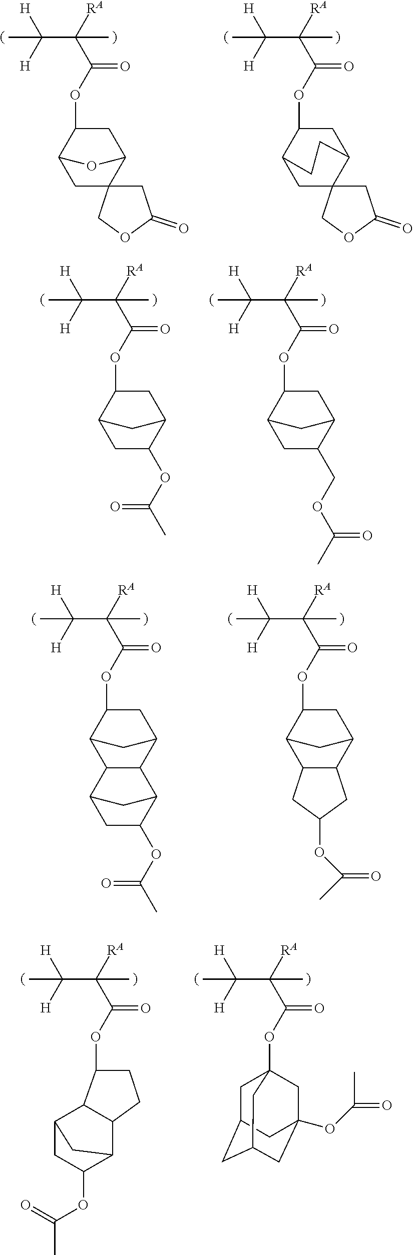

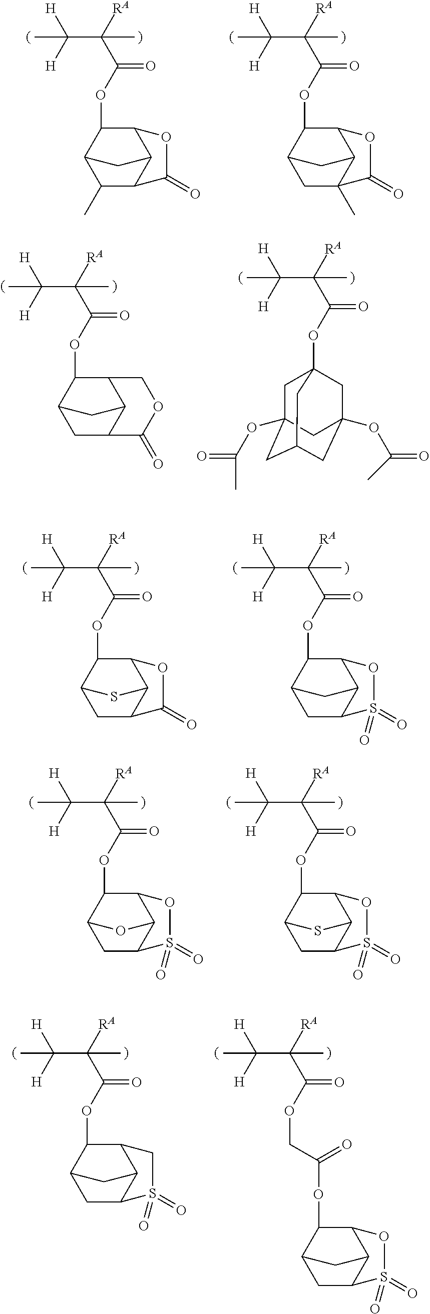

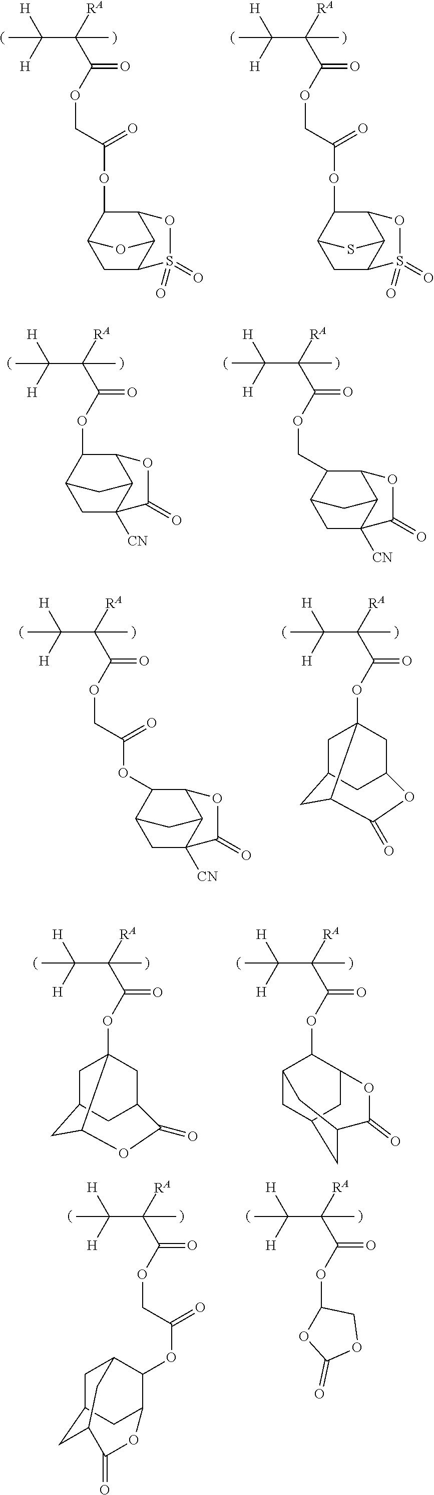

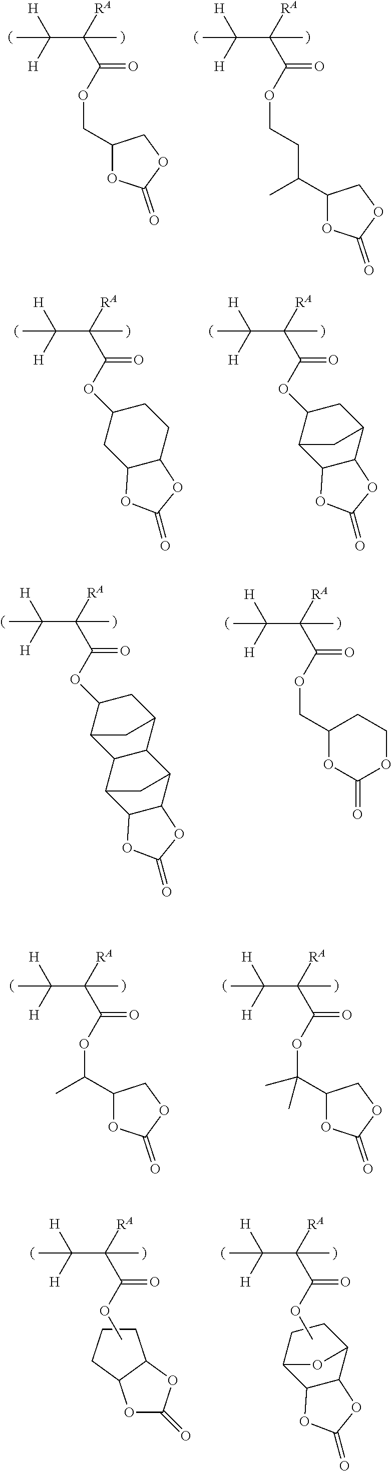

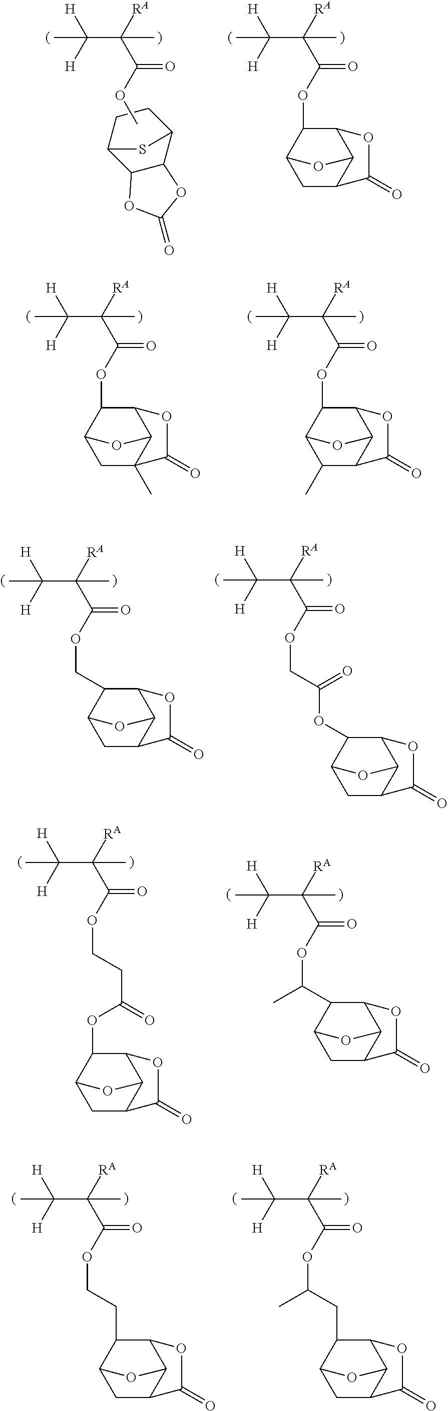

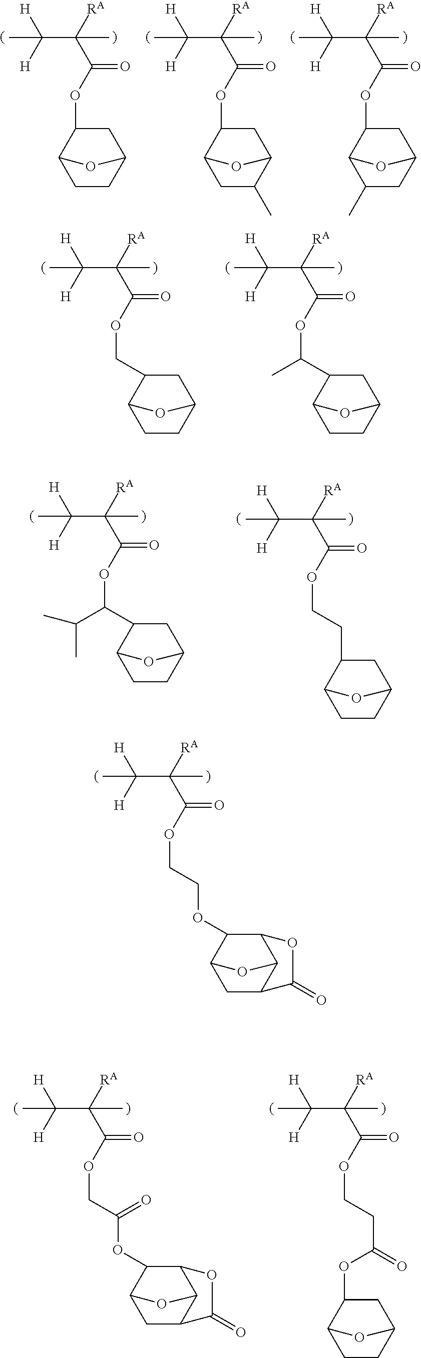

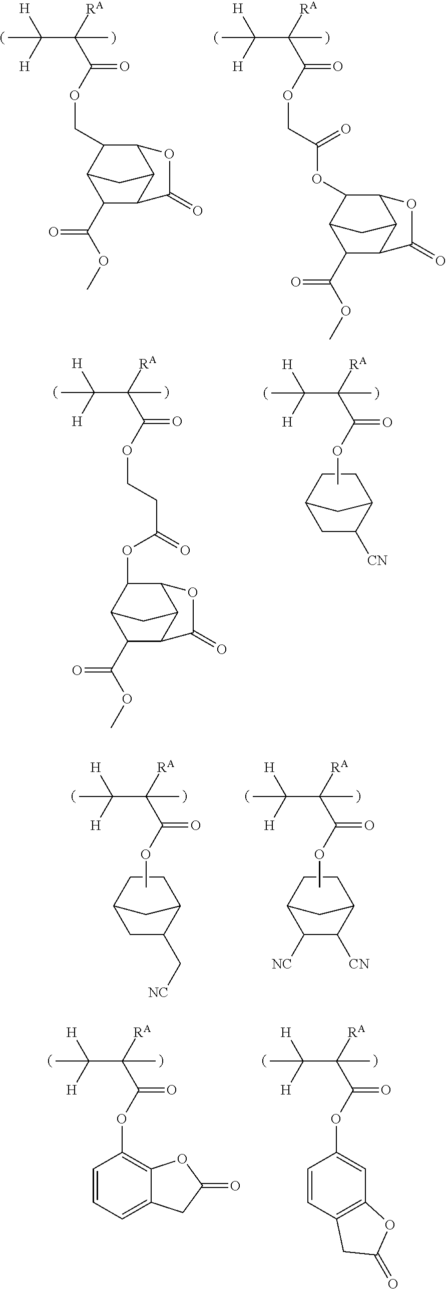

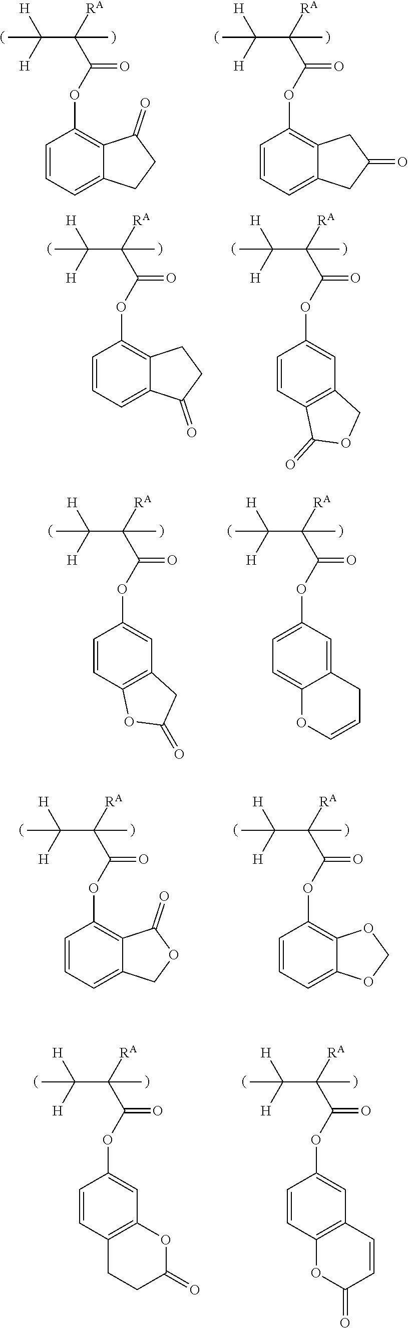

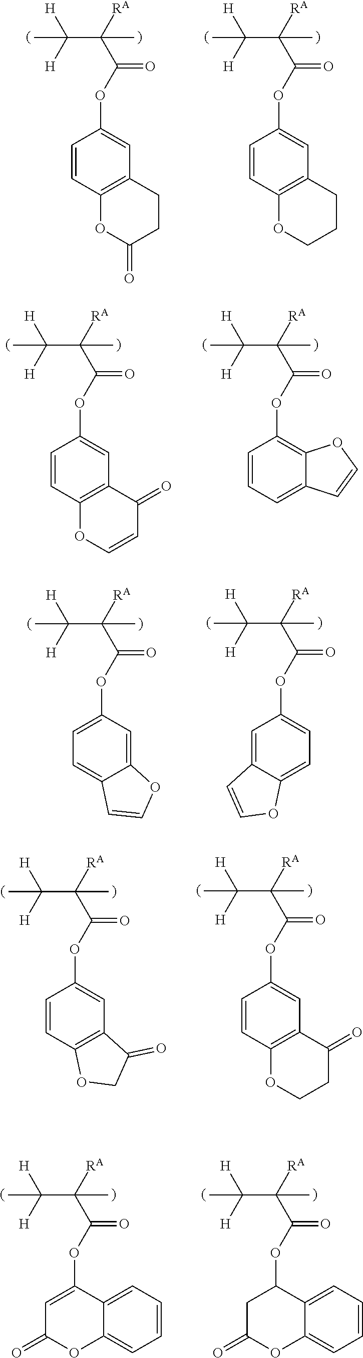

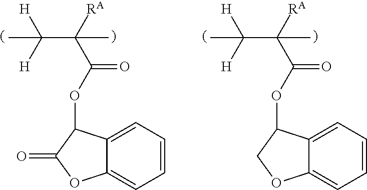

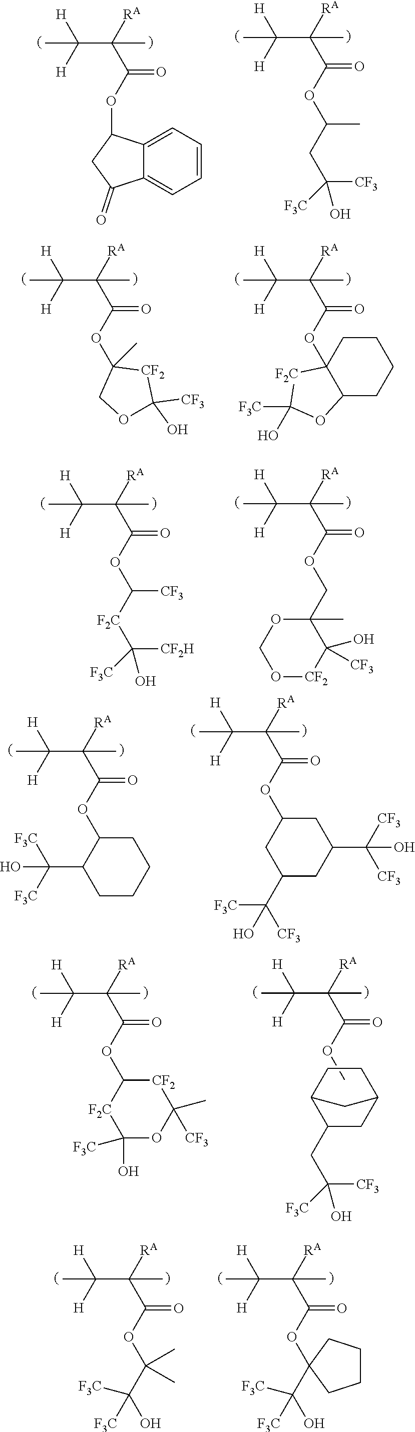

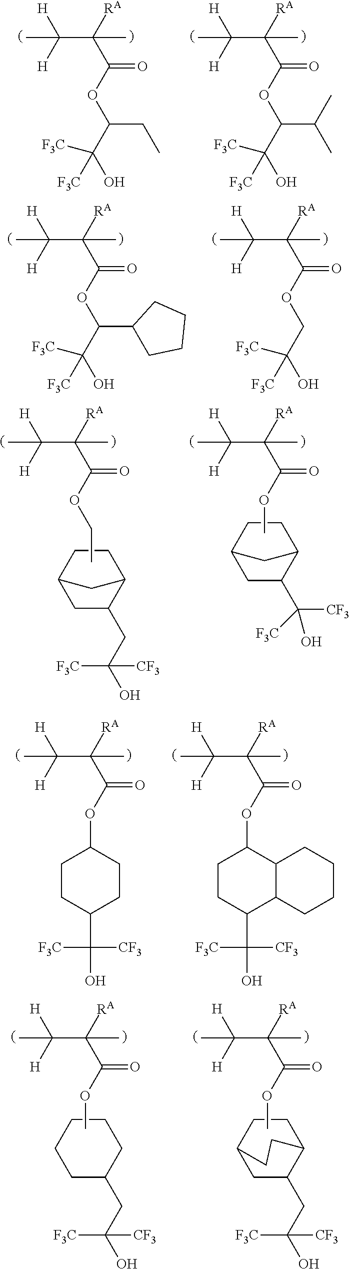

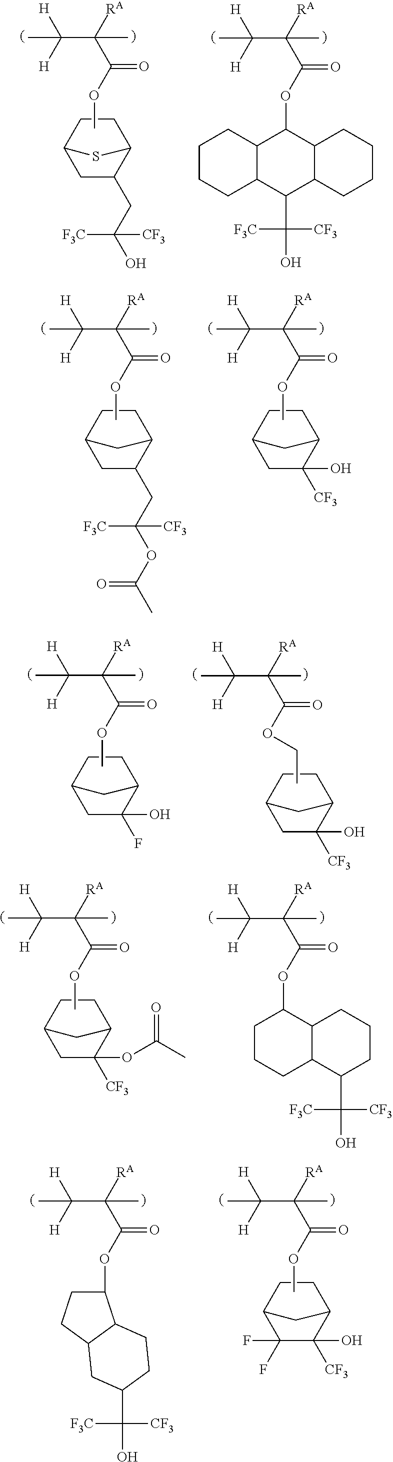

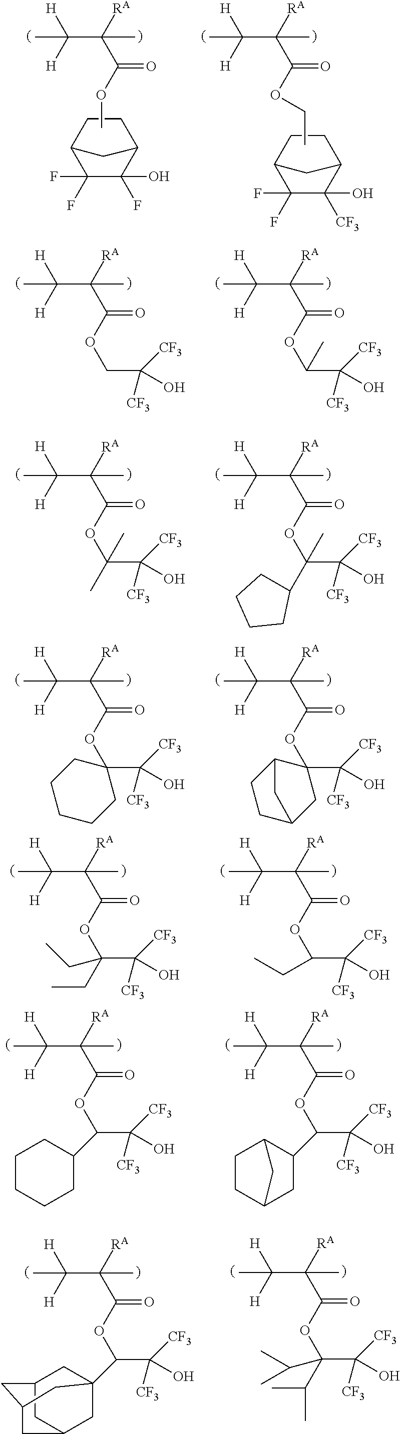

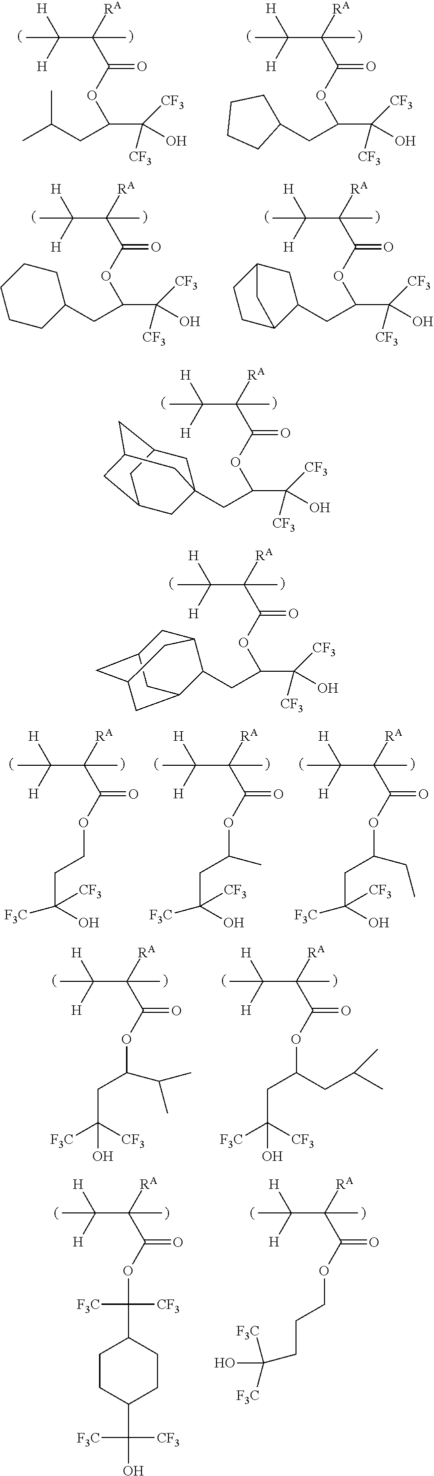

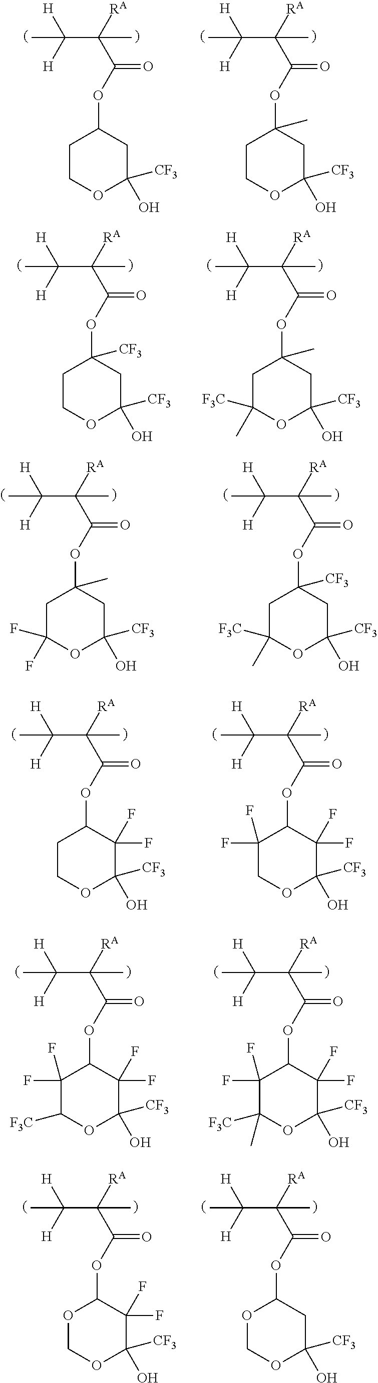

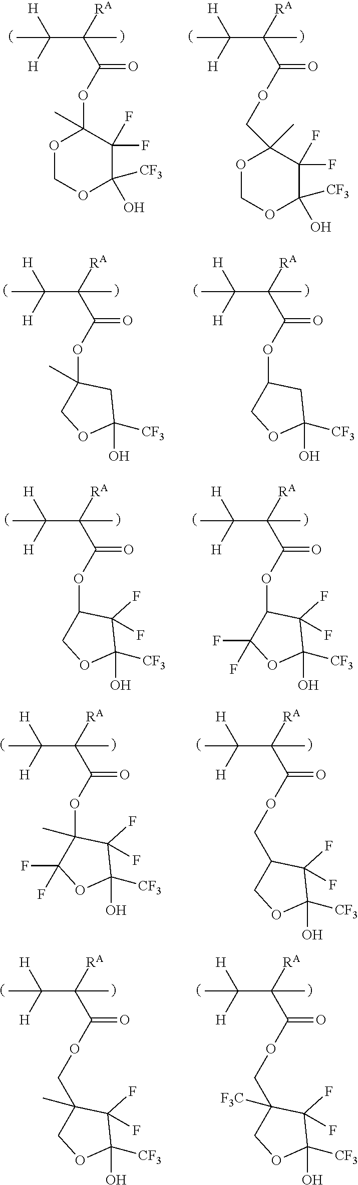

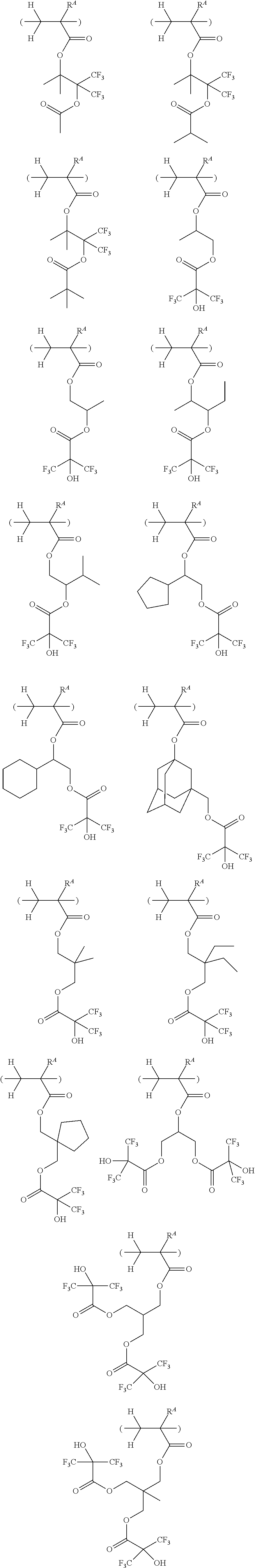

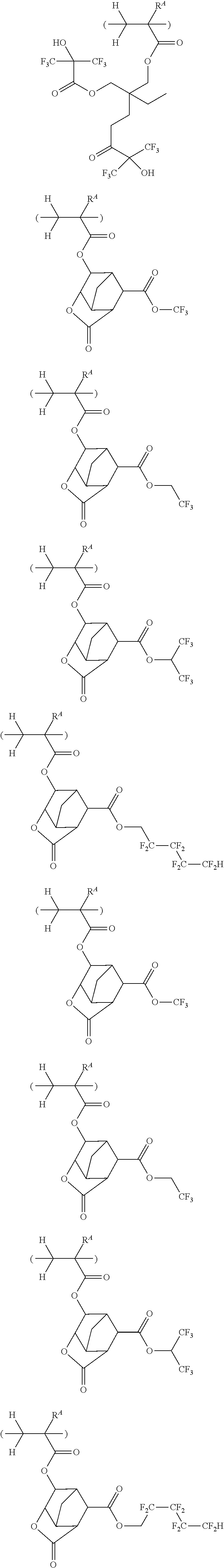

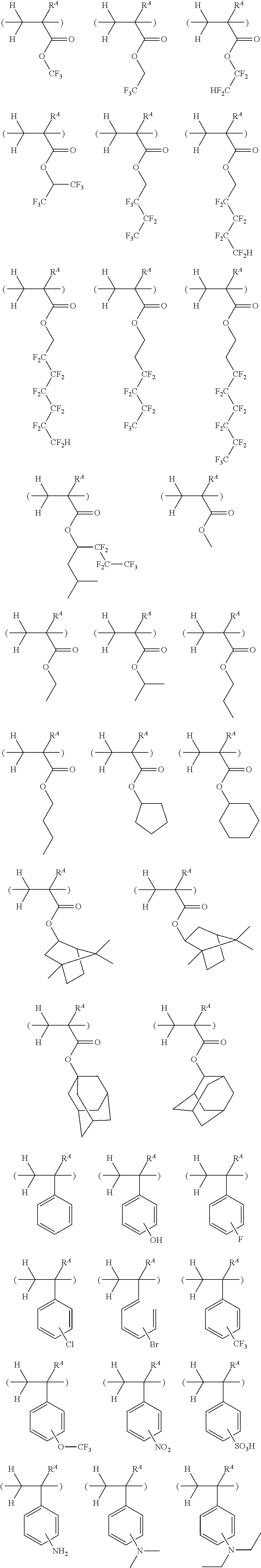

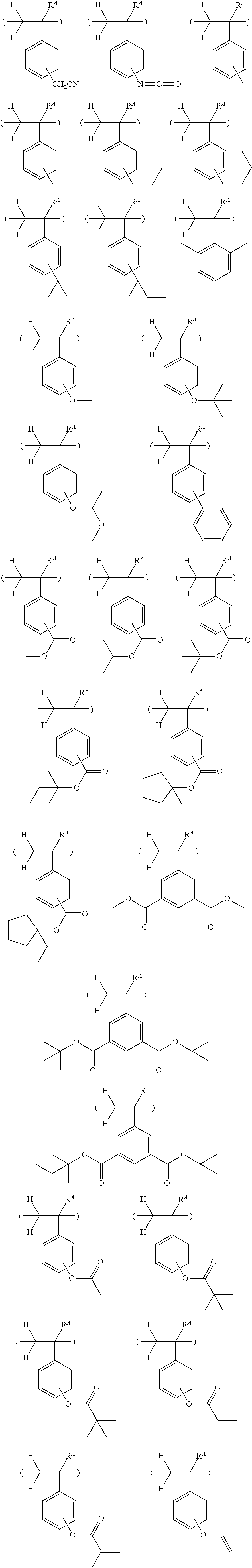

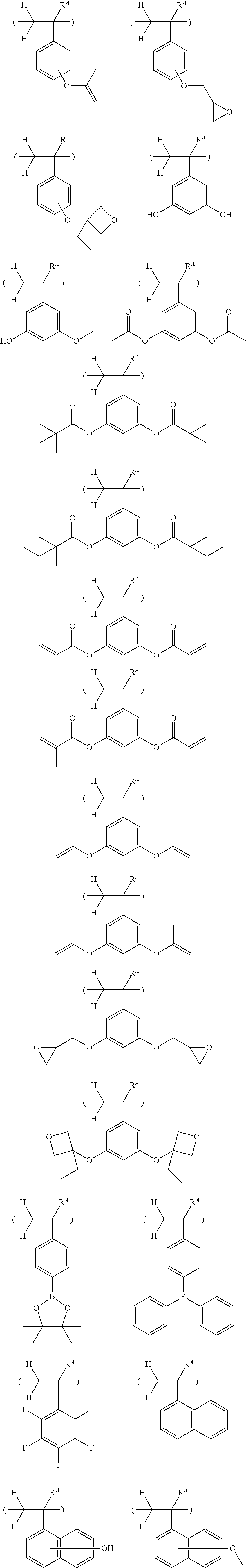

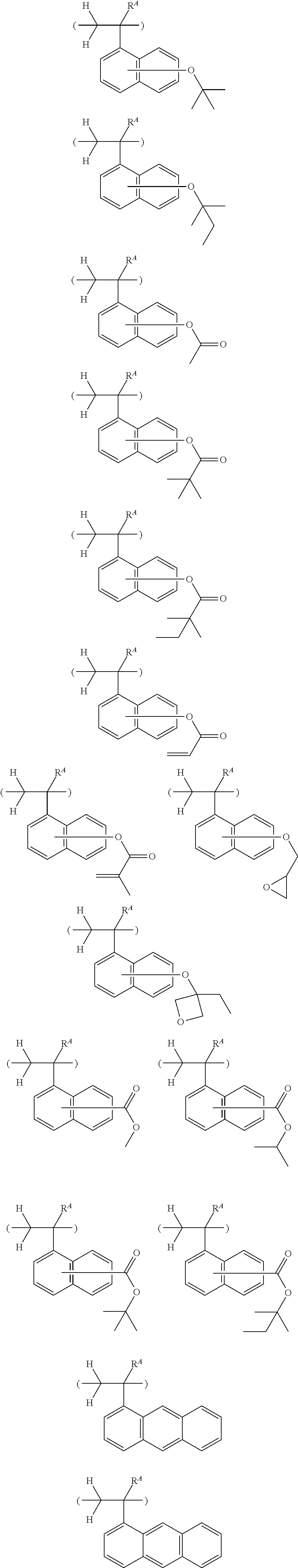

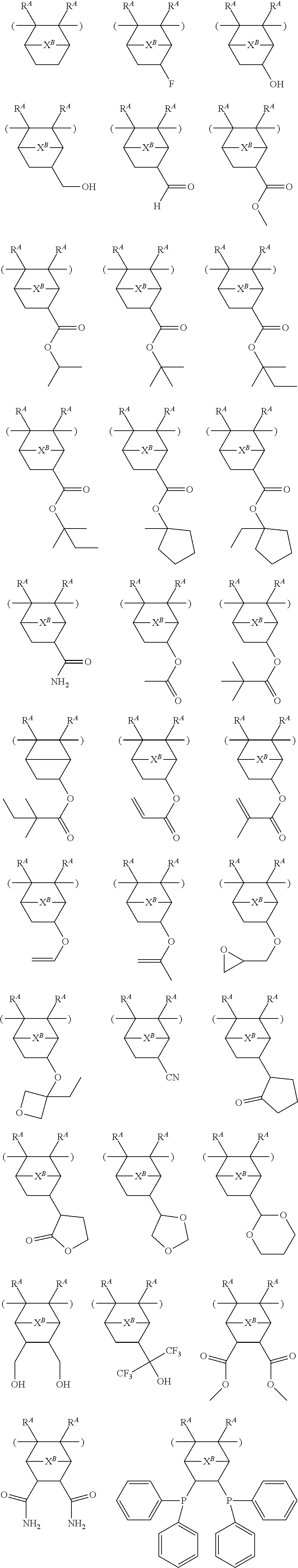

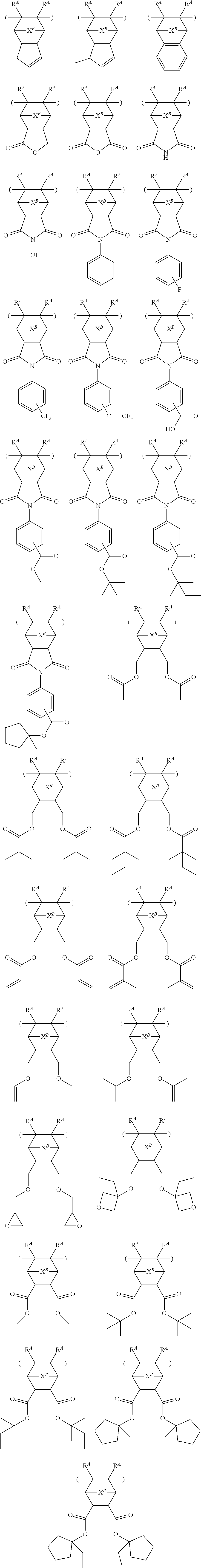

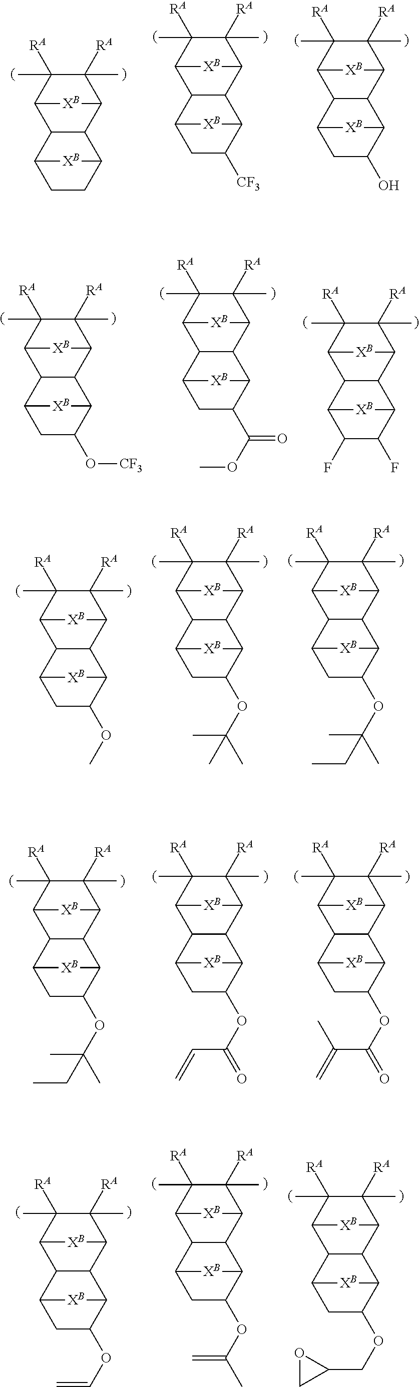

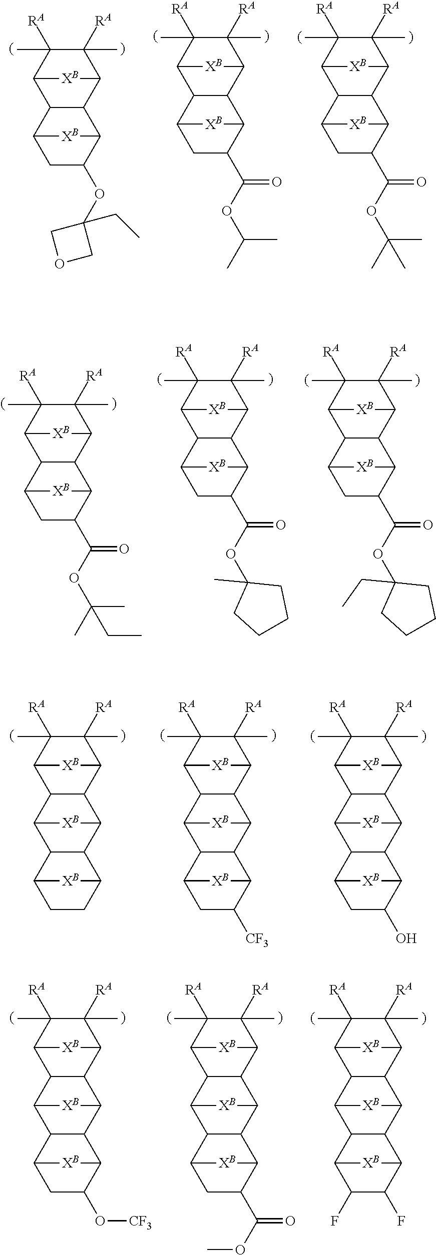

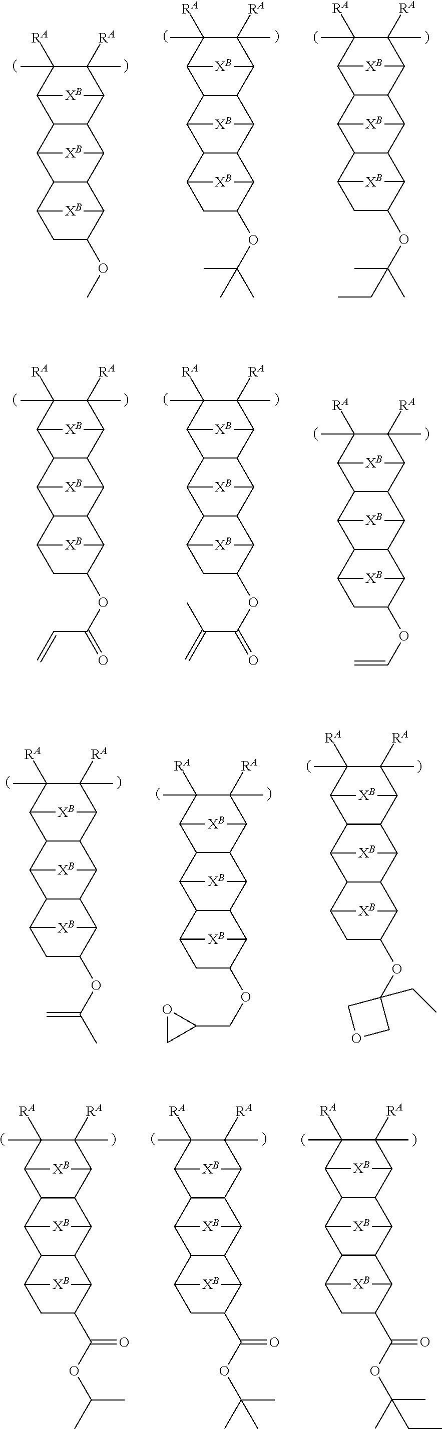

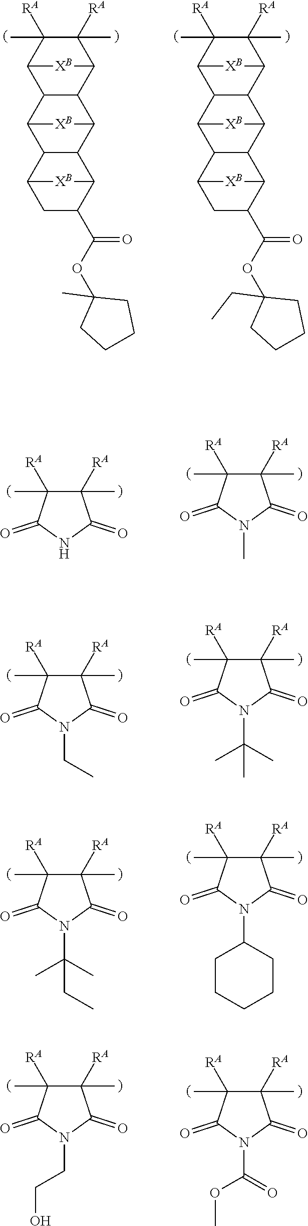

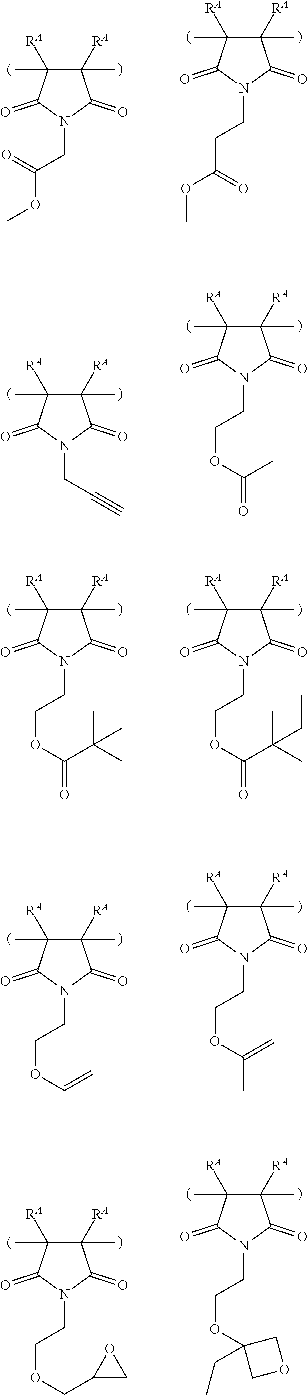

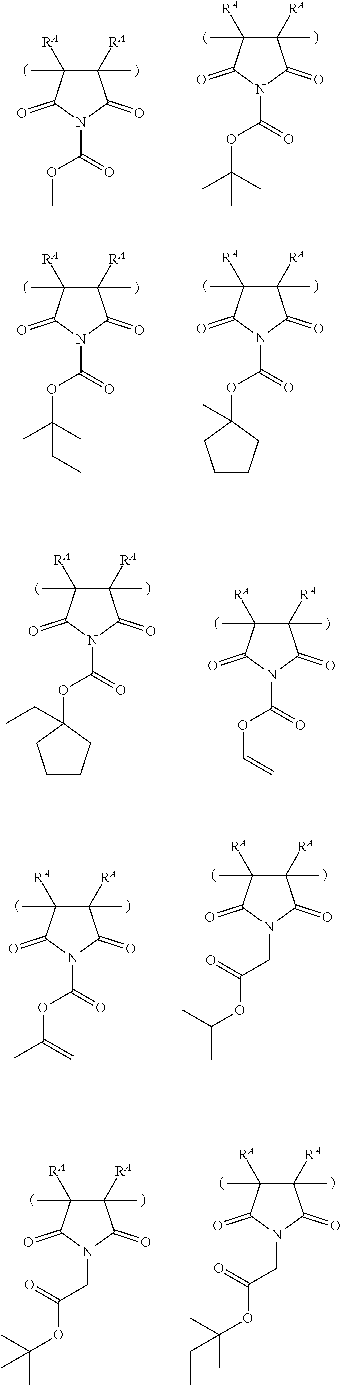

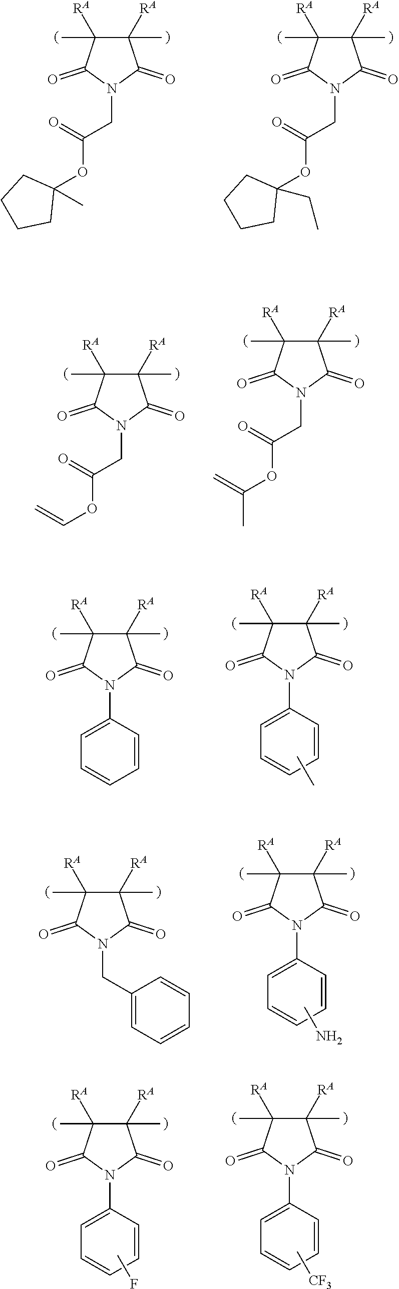

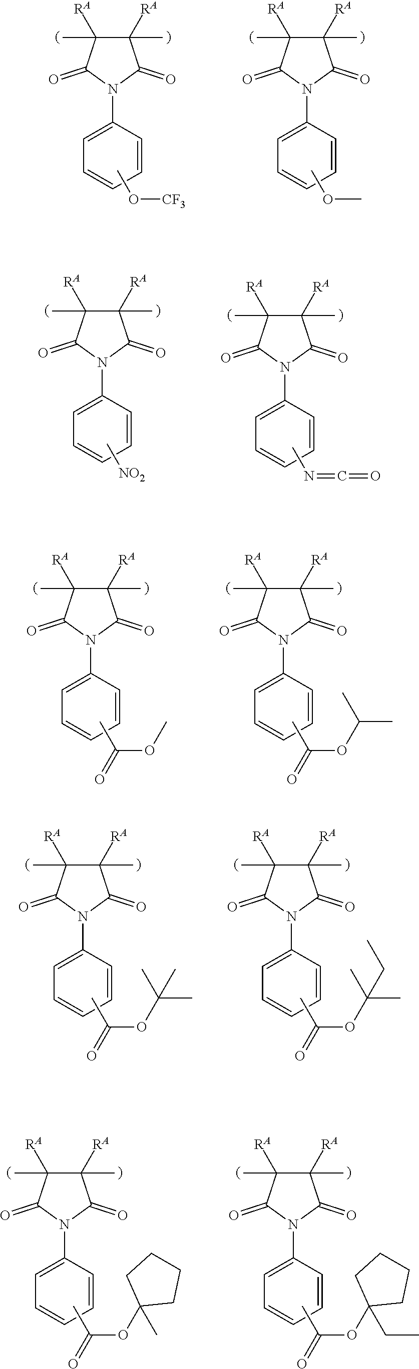

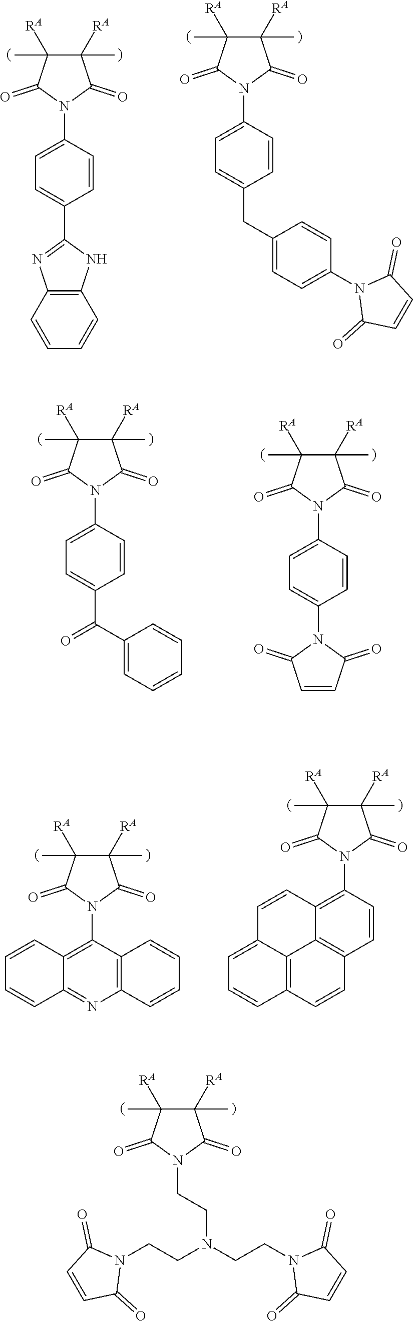

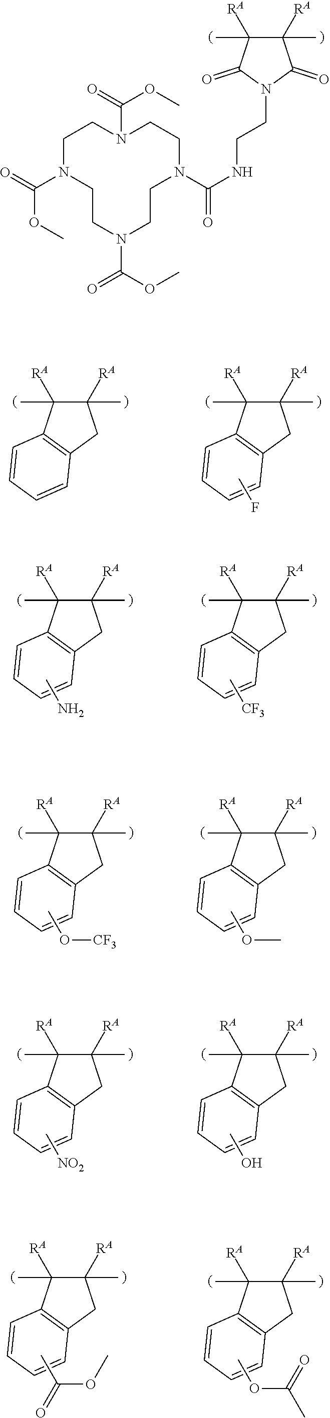

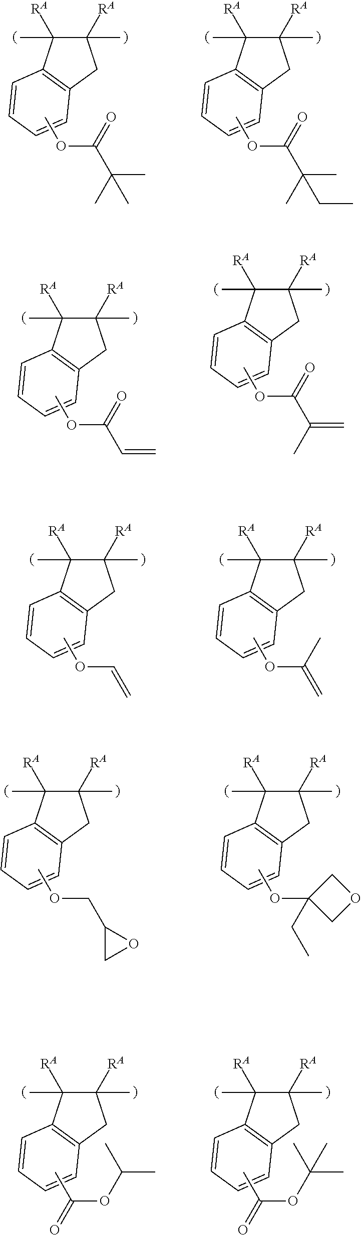

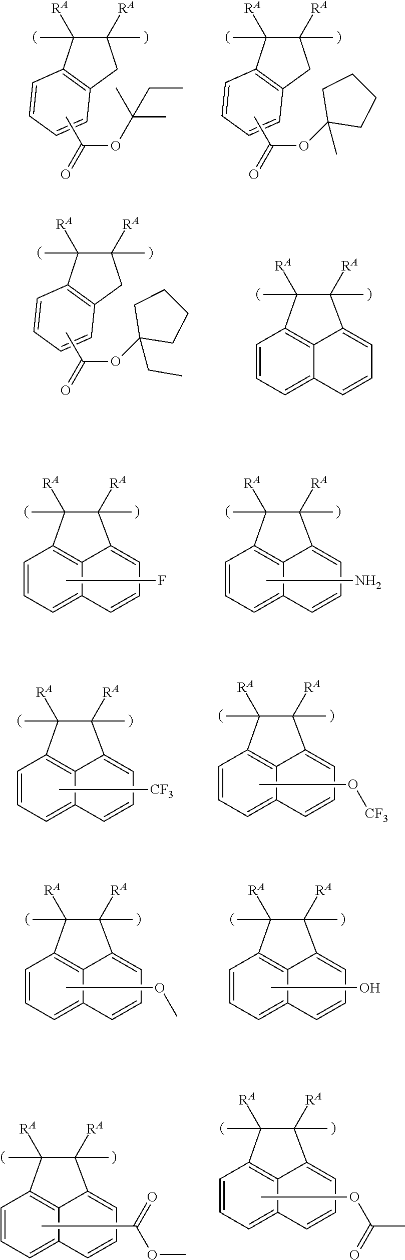

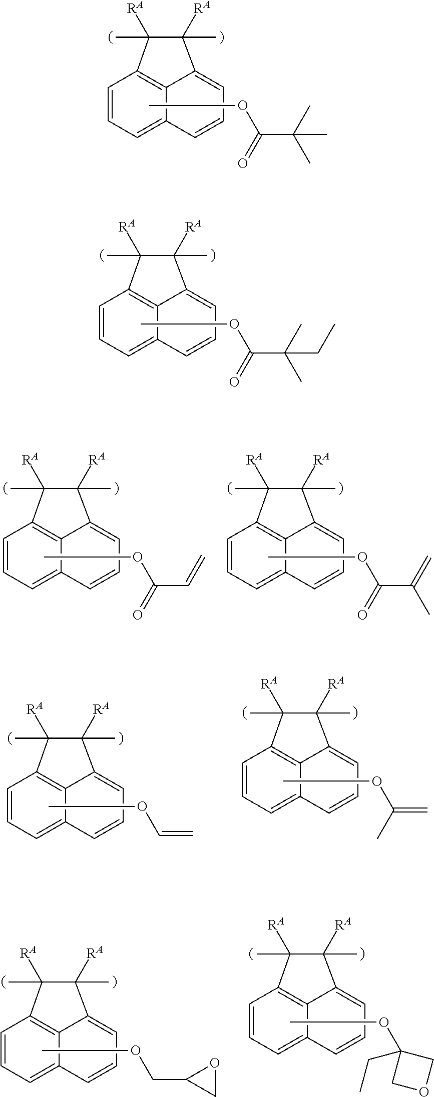

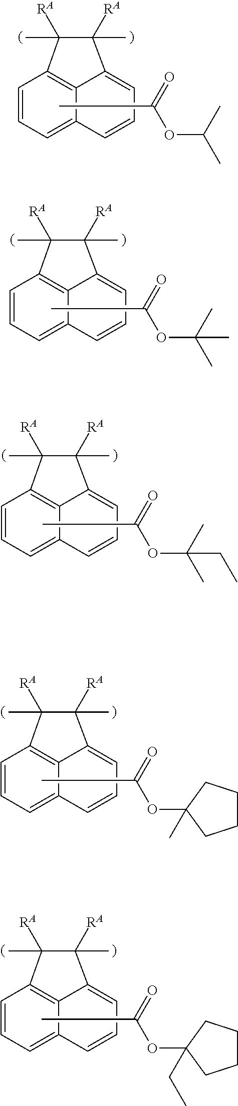

In the carboxy group-containing polymer, the carboxy group-containing recurring unit and another recurring unit are preferably present in a content ratio (molar ratio) of carboxy group-containing recurring unit:another recurring unit=10:90 to 90:10, more preferably 15:85 to 85:15, and still more preferably 20:80 to 80:20.

The carboxy group-containing polymer preferably has a weight average molecular weight (Mw) of 1000 to 500000, and more preferably 3000 to 100000. In the invention, Mw represents a value measured by gel permeation chromatography (GPC) versus polystyrene standards using tetrahydrofuran (THF) as a solvent.

If the carboxy group-containing polymer has a wide molecular weight distribution or dispersity (Mw/Mn), which indicates the presence of lower and higher molecular weight polymer fractions, there is a possibility that foreign matter is left on the pattern or the pattern profile is degraded after exposure. The influences of Mw and Mw/Mn become stronger as the pattern rule becomes finer. Therefore, the carboxy group-containing polymer should preferably have a narrow dispersity (Mw/Mn) of 1.0 to 2.0 in order to provide a resist composition suitable for micropatterning to a small feature size.

Examples of the method of synthesizing the carboxy group-containing polymer include a method in which monomers corresponding to the foregoing recurring units are dissolved in an organic solvent, a radical polymerization initiator is added thereto, and the resulting mixture is heated for polymerization.

Examples of the organic solvent used in the polymerization reaction include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the polymerization initiator added is preferably 0.01 to 25 mol % per total amount of monomers to be polymerized. The reaction temperature is preferably 50 to 150° C., and more preferably 60 to 100° C. The reaction time is preferably 2 to 24 hours, and more preferably 2 to 12 hours from the viewpoint of production efficiency.

The polymerization initiator may be added to the monomer solution before supply to a reaction vessel, or an initiator solution may be prepared separately from the monomer solution and each solution may be supplied to a reaction vessel independently. Radicals generated from the initiator during waiting time may promote a polymerization reaction to generate an ultrahigh polymer. Therefore, from the viewpoint of quality control, each of the monomer solution and the initiator solution is preferably prepared and added dropwise independently. The acid labile group introduced into the monomer may be used as it is, or may be protected or partially protected after polymerization. Further, a known chain transfer agent such as dodecyl mercaptan or 2-mercaptoethanol may be used in combination for adjusting the molecular weight. In this case, the amount of such a chain transfer agent added is preferably 0.01 to 20 mol % per total amount of monomers to be polymerized.

The amount of each monomer in the monomer solution is to be appropriately set, for example, so as to achieve the foregoing preferred content ratio of the recurring unit.

In the resist composition, the hypervalent iodine compound and the carboxy group containing polymer are preferably present in a content ratio such that the molar ratio of the hypervalent iodine compound to the carboxylic acid-containing recurring unit in the polymer is 10:90 to 90:10, more preferably 20:80 to 80:20, and still more preferably 30:70 to 70:30. The hypervalent iodine compounds may be used alone or in admixture of two or more thereof. The carboxy group-containing polymer may be used alone or in admixture of two or more having different composition ratios and different values of Mw and/or Mw/Mn.

### [Another Hypervalent Iodine Compound]

The resist composition of the invention may contain hypervalent iodine compounds having the formula (3) (hereinafter, also referred to as another hypervalent iodine compound). By adding another hypervalent iodine compound, the reactivity to light can be controlled to adjust the sensitivity.

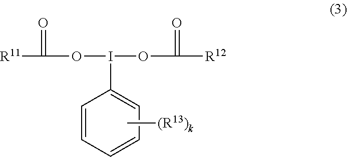

In the formula (3), k is an integer of 0 to 5.

In the formula (3), R¹¹ and R¹² are each independently halogen or a C₁-C₁₀ hydrocarbyl group which may contain a heteroatom. R¹¹ and R¹² may bond together to form a ring with the carbon atoms to which they are attached, and atoms between the carbon atoms. Examples of the halogen include fluorine, chlorine, bromine and iodine atoms. The C₁-C₁₀ hydrocarbyl group may be saturated or unsaturated and straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₁₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decanyl and adamantyl groups; C₂-C₁₀ alkenyl groups such as vinyl and allyl groups; C₆-C₁₀ aryl groups such as phenyl and naphthyl groups; and combinations thereof. Also included are hydrocarbyl groups in which some or all of the hydrogen atoms are substituted by a moiety containing a heteroatom such as oxygen, sulfur, nitrogen, or halogen, and some constituent —CH₂— is replaced by a moiety containing a heteroatom such as oxygen, sulfur, or nitrogen, so that the group may contain hydroxy, cyano, halogen, carbonyl, ether bond, thioether bond, ester bond, sulfonic ester bond, carbonate bond, carbamate bond, lactone ring, sultone ring, or carboxylic anhydride (—C(═O)—O—C(═O)—). R¹¹ and R¹² are each preferably a C₁-C₄ hydrocarbyl or C₁-C₄ fluorinated hydrocarbyl group, and more preferably a C₁-C₄ hydrocarbyl group.

In the formula (3), R¹³ is halogen, or a C₁-C₄₀ hydrocarbyl group which may contain a heteroatom. Examples of the halogen include fluorine, chlorine, bromine and iodine atoms. The C₁-C₄₀ hydrocarbyl group may be saturated or unsaturated and straight, branched or cyclic. Examples thereof include C₁-C₄₀ alkyls such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decanyl, adamantyl and adamantylmethyl groups; C₆-C₄₀ aryl groups such as phenyl, naphthyl and anthracenyl groups; and combinations thereof. Also included are hydrocarbyl groups in which some or all of the hydrogen atoms are substituted by a moiety containing a heteroatom such as oxygen, sulfur, nitrogen, or halogen, and some constituent —CH₂— is replaced by a moiety containing a heteroatom such as oxygen, sulfur, or nitrogen, so that the group may contain hydroxy, cyano, halogen, carbonyl, ether bond, thioether bond, ester bond, sulfonic ester bond, carbonate bond, carbamate bond, lactone ring, sultone ring, or carboxylic anhydride (—C(═O)—O—C(═O)—). When k is 2, 3, 4 or 5, R¹³ may be the same or different, and a plurality of R¹³ may bond together to form a ring with the aromatic ring carbon atoms to which they are attached.

Examples of the another hypervalent iodine compound are shown below, but not limited thereto.

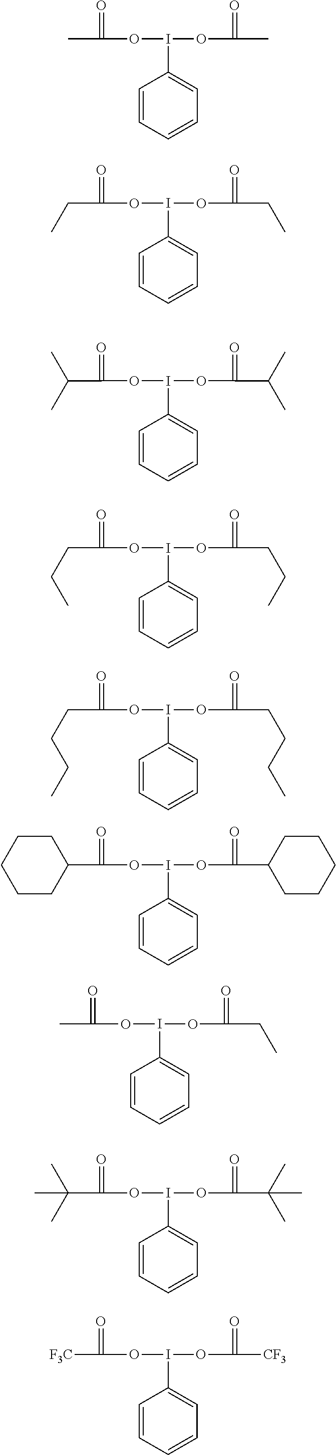

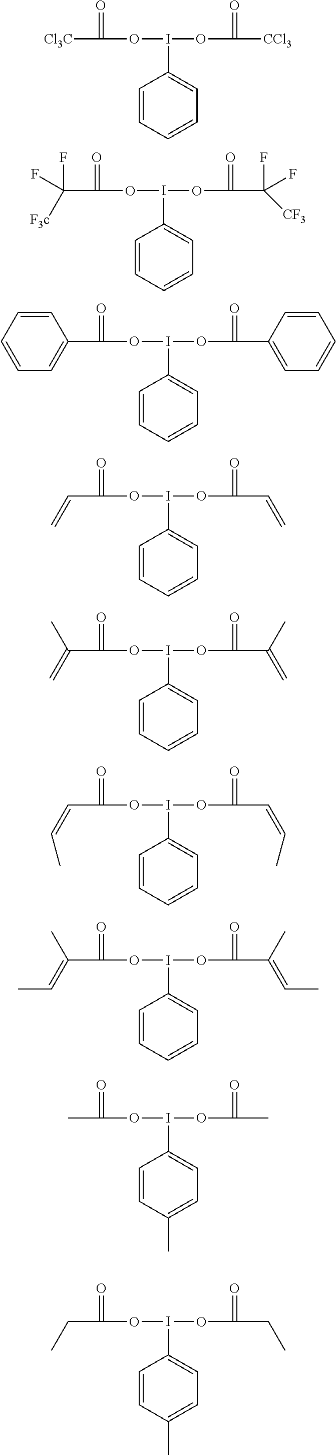

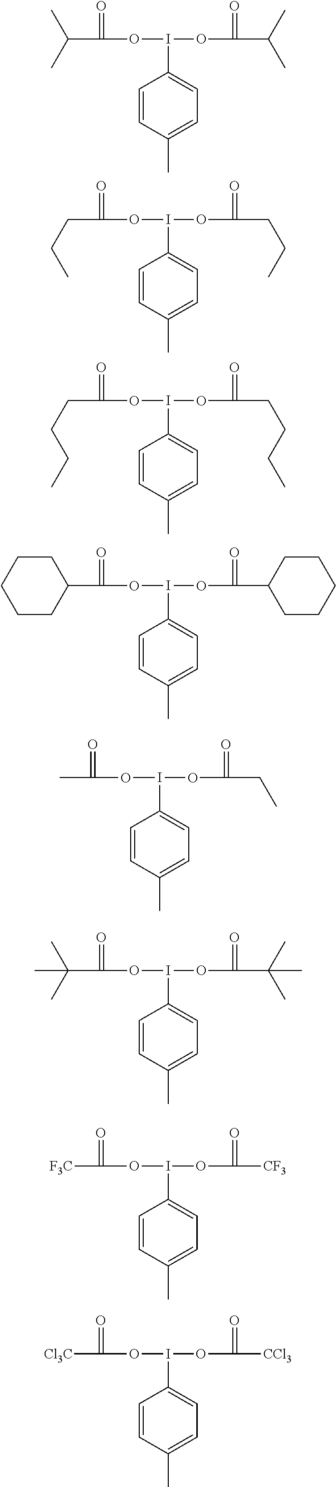

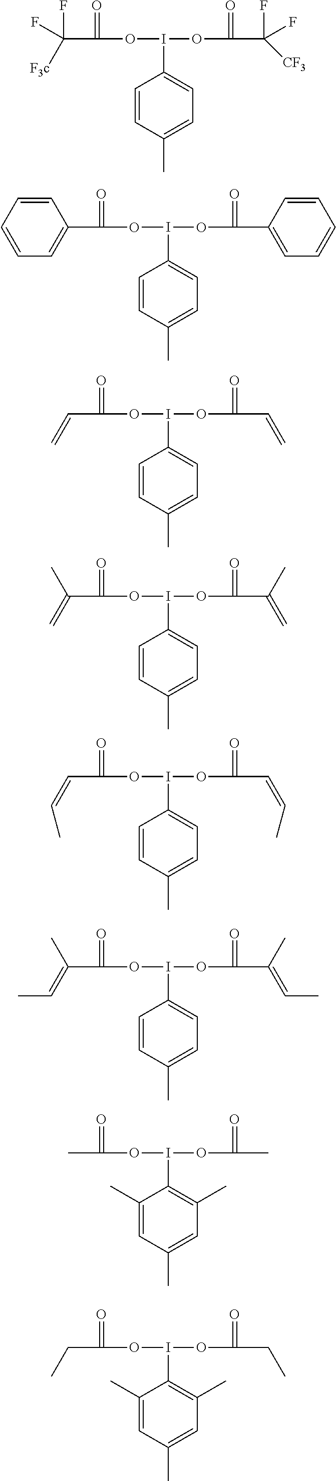

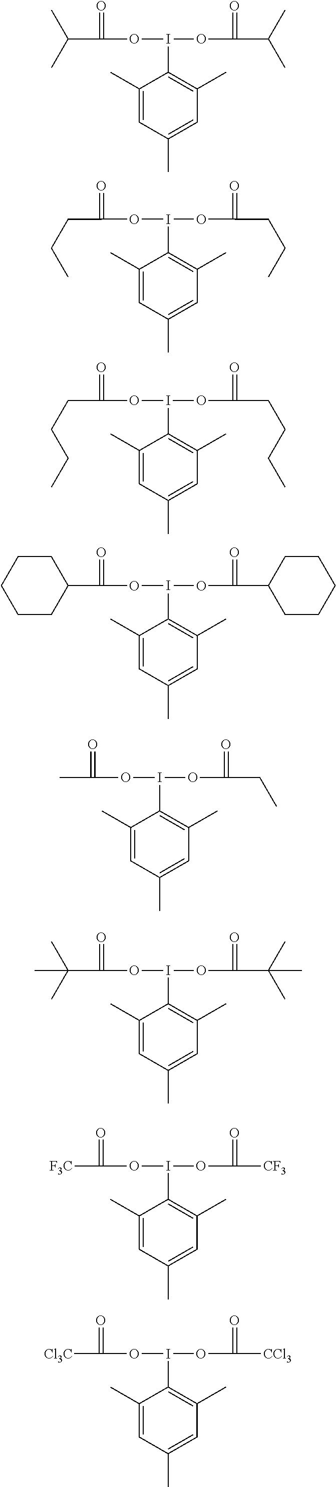

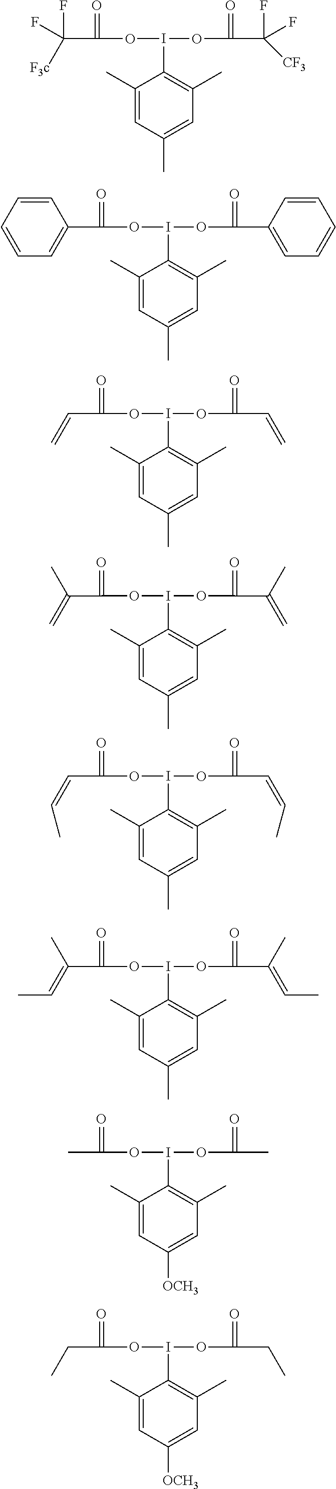

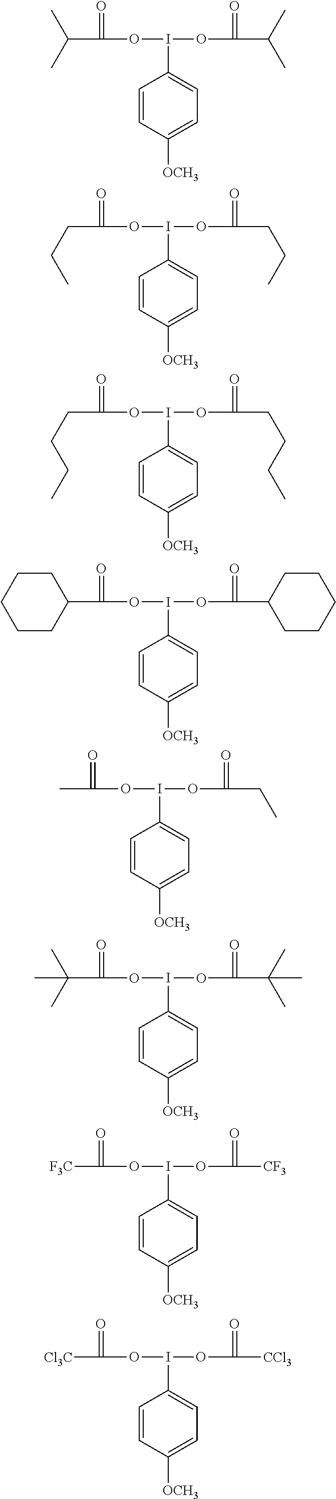

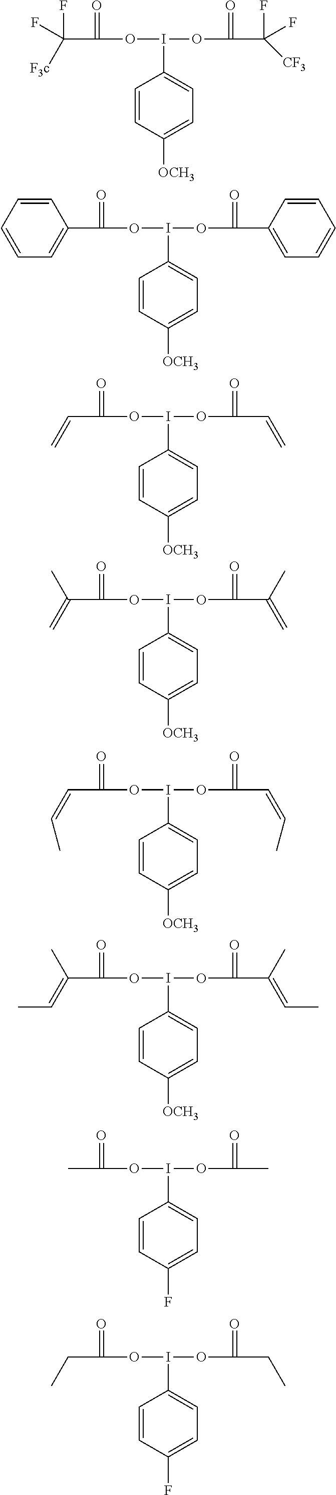

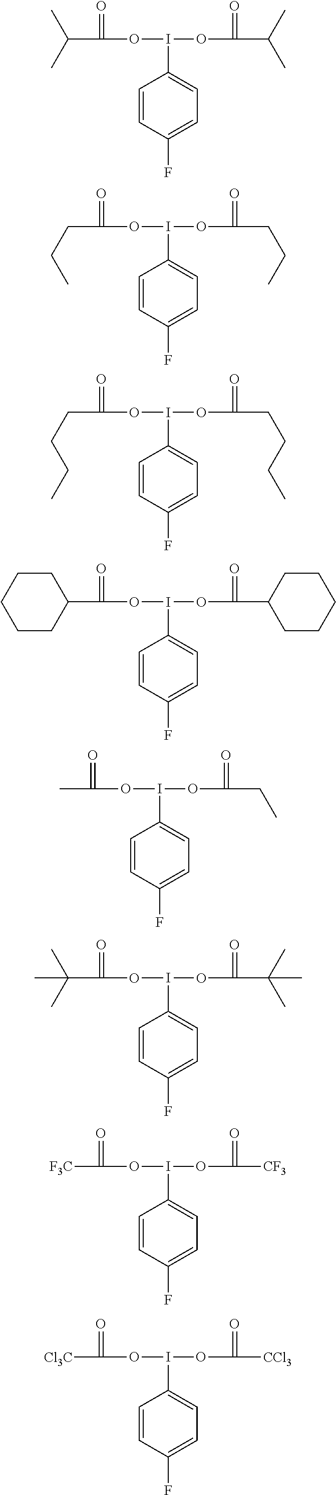

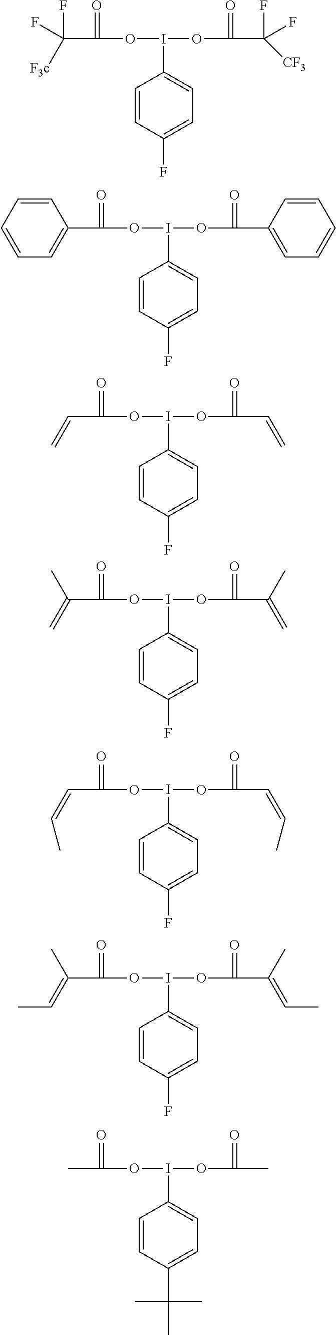

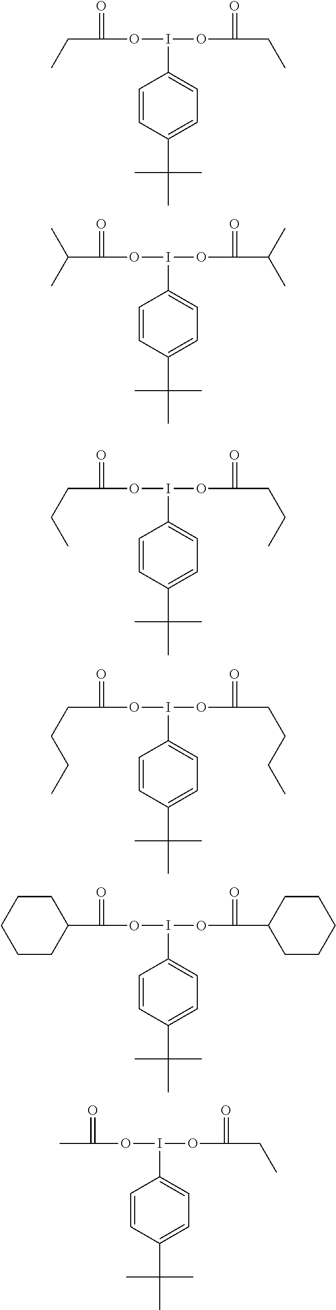

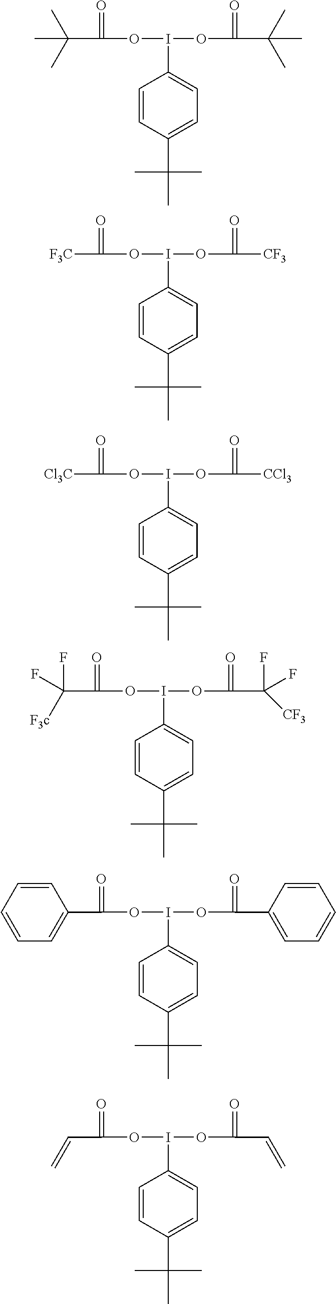

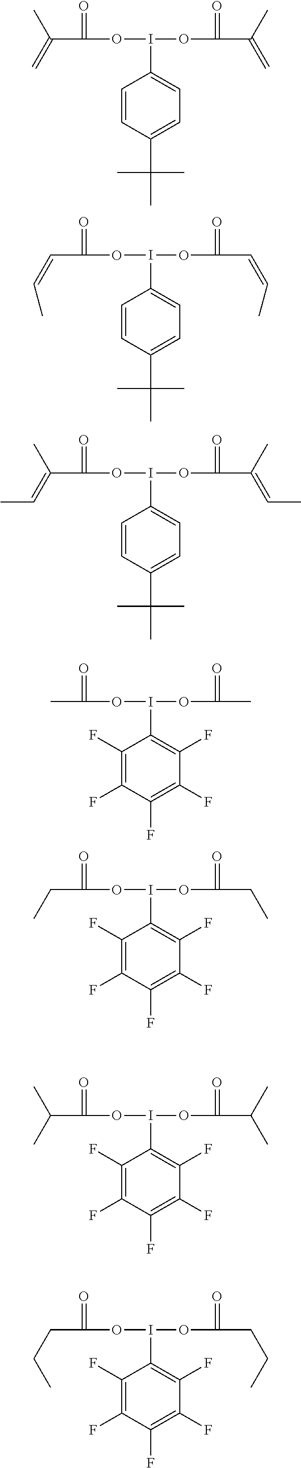

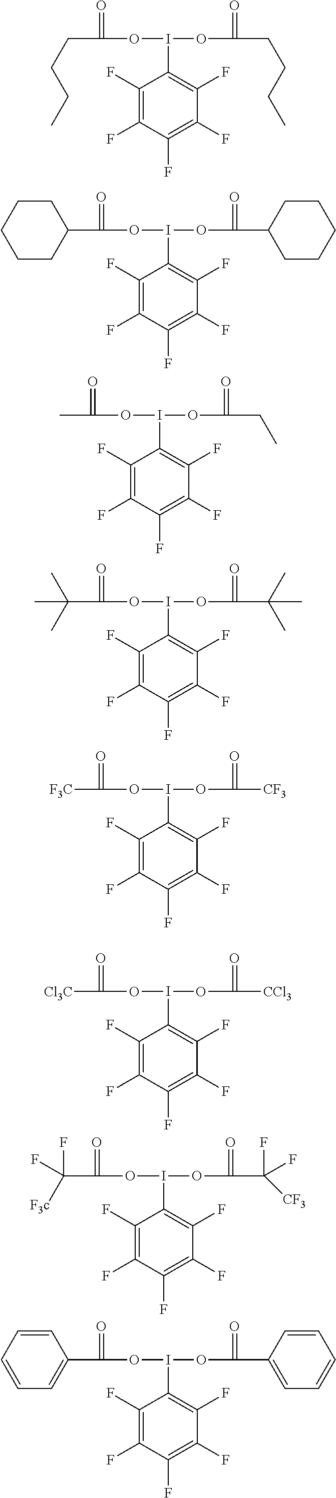

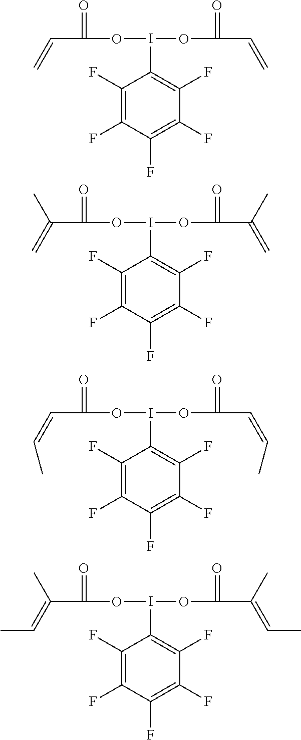

In the resist composition of the invention, another hypervalent iodine compound is preferably present in a content ratio such that the molar ratio of the total of the hypervalent iodine compound having the formula (1) and the another hypervalent iodine compound to the carboxylic acid-containing recurring unit in the polymer is 10:90 to 90:10, more preferably 20:80 to 80:20, and still more preferably 30:70 to 70:30. The another hypervalent iodine compound is preferably present in a content ratio such that the molar ratio of another hypervalent iodine compound:hypervalent iodine compound having the formula (1) is 1:99 to 99:1, and more preferably 1:99 to 50:50. The another hypervalent iodine compound may be used alone or in admixture of two or more thereof.

### [Solvent]

The resist composition contains a solvent. The solvent is not particularly limited as long as the hypervalent iodine compound having the formula (1), another hypervalent iodine compound, and other components described below are dissolvable therein and a film can be formed from the resulting solution. The solvent is preferably an organic solvent, and examples of the organic solvent include ketones such as cyclohexanone, methyl-2-n-pentyl ketone and methyl isoamyl ketone; alcohols such as 3-methoxybutanol, 3-methyl-3-methoxybutanol, 1-methoxy-2-propanol, 1-ethoxy-2-propanol, diacetone alcohol, 4-methyl-2-pentanol and methyl 2-hydroxyisobutyrate; ethers such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate and propylene glycol mono-tert-butyl ether acetate; carboxylic acids such as formic acid, acetic acid and propionic acid; lactones such as γ-butyrolactone; and mixtures thereof.

In the resist composition, the solvent is preferably present in such amounts that the resist composition may have a solids concentration of 0.1 to 20 wt %, more preferably 0.1 to 15% wt %, even more preferably 0.1 to 10 wt %. As used herein, the term solids is a general term for all components in the resist composition excluding the solvent. The solvents may be used alone or in admixture of two or more thereof.

The resist composition may further contain a surfactant. The surfactant is preferably a fluorine-based and/or silicon-based surfactant. Exemplary surfactants are described, for example, in US 2008/0248425, paragraph [0276]. Also useful are surfactants other than the fluorine-based and/or silicon-based surfactants, as described, for example, in US 2008/0248425, paragraph [0280].

When used, the surfactant is preferably present in an amount of 0.0001 to 2 wt % based on the overall solids. The surfactants may be used alone or in admixture of two or more thereof.

The resist composition of the invention may further contain at least one selected from a radical scavenger and a crosslinker. When added, the radical scavenger and/or crosslinker are effective for controlling photo-reaction and adjusting sensitivity during photolithography.

Suitable radical scavengers include hindered phenols, quinones, hindered amines, and thiol compounds. Specifically, exemplary hindered phenols include dibutylhydroxytoluene and 2,2′-methylenebis(4-methyl-6-tert-butylphenol). Exemplary quinones include 4-methoxyphenol (or methoquinone) and hydroquinone. Exemplary hindered amines include 2,2,6,6-tetramethylpyperidine and 2,2,6,6-tetramethylpyperidine-N-oxy radical. Exemplary thiol compounds include dodecanethiol and hexadecanethiol.

When used, the radical scavenger is preferably present in an amount of 0.01 to 10 wt % based on the overall solids. The radical scavengers may be used alone or in admixture of two or more thereof.

Exemplary crosslinkers include compounds having a functional group having a carbon-carbon unsaturated bond, such as a vinyl group, a (meth)acrylate group, an allyl group, an alkynyl group and an aromatic ring. Exemplary compounds having a vinyl group include chain alkenes, branched alkenes and cyclic alkenes which may be substituted. Exemplary compounds having a (meth)acrylate group include acrylic acids, methacrylic acids, acrylic acid esters and methacrylic acid esters which may be substituted. Exemplary compounds having an allyl group include allyl alcohols, allyl ethers, allyl esters, allyl amides, allyl amines and allyl group-containing isocyanurates which may be substituted. Exemplary compounds having an alkynyl group include chain alkynes, branched alkynes, cyclic alkynes, alkynyl alcohols, alkynyl ethers, alkynyl esters, alkynyl amides, alkynyl amines and alkynyl group-containing isocyanurates which may be substituted. Exemplary compounds having an aromatic ring include arenes, heteroarenes, styrene, stilbene, phenylacetylene, acenaphthylene and chalcone which may be substituted. The crosslinker may have only one of the functional groups, or two or more of the functional groups. The number of the functional groups contained in the crosslinker is preferably 1 or more and 10 or less, more preferably 2 or more and 8 or less.

When used, the crosslinker is preferably present in an amount of 0.01 to 50 wt % based on the overall solids. The crosslinkers may be used alone or in admixture of two or more thereof.

As described above, the resist composition contains the hypervalent iodine compound and the carboxy group-containing polymer as principal components, but not a base polymer containing an acid labile group and a photoacid generator as used in conventional chemically amplified resist compositions. Nevertheless, this resist composition works such that exposure to EB or EUV produces a difference in solubility between exposed regions and unexposed regions to form positive tone or negative tone pattern. Although its mechanism is not well understood, the following mechanism is presumed.

The hypervalent iodine compound having the formula (1) is a three-coordinate compound containing carboxylate ligands. When such a three-coordinate iodine compound is mixed with a carboxylic acid, replacement of carboxylate ligands takes place as equilibration reaction. If the original carboxylate ligands are removed by any suitable means, a hypervalent iodine compound having new ligands is created. For example, if 1-acetoxy-1,2-benziodoxol-3-(1H)-one which is relatively readily available as the hypervalent iodine compound is mixed with a carboxylic acid having a high molecular weight, and the resulting low-boiling acetic acid is removed, then ligand exchange is completed. Here, when the carboxylic acid is a polymer, the polymers are bonded to the hypervalent iodine compound to form a high molecular weight hypervalent iodine compound.

The high molecular weight hypervalent iodine compound is generated during film formation. This is because even if synthesized in advance, such a high molecular weight hypervalent iodine compound is insoluble in most organic solvents, so that it is impossible to prepare their solution. This is presumed to be because the hypervalent iodine compound, which is originally low in solvent solubility due to the large polarization, further deteriorates in solubility by using a carboxylic acid-containing polymer that is a high molecular weight compound as a ligand. Therefore, it is desirable that the original low-molecular-weight carboxylic acid is removed during film formation and a subsequent bake step to complete a ligand exchange reaction and form a resist film in the step.

The resist film thud formed is presumed to contain a polymer in which a carboxy group-containing polymer and a hypervalent iodine compound are bonded. The polymer is presumed to contain a recurring unit having the formula (4):

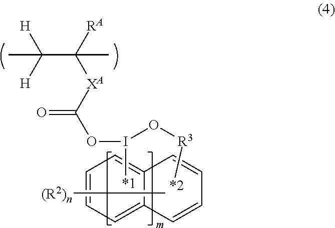

wherein m, n, R², R³, *1, *2, R^{A} and X^{A} are as defined above.

The inventive resist film thus formed on the substrate undergoes a change in polarity due to photodecomposition of the hypervalent iodine compound being a principal component of the resist film, and is patterned by the development step. A positive-tone or negative-tone pattern can be formed by appropriately selecting a developer.

From the foregoing presumption, the inventive resist composition is a non-chemically amplified resist composition. The inventive resist composition does not need a base polymer containing an acid labile group and a photoacid generator as used in conventional chemically amplified resist compositions. Therefore, a small size pattern can be resolved without an adverse effect (for example, image blur) due to acid diffusion.

The resist composition of the invention is very useful particularly in EUV lithography. This is because the resist composition of the invention has an iodine atom which has a high ability to absorb EUV light, and the hypervalent iodine compound having the formula (1) has only one carboxylate ligand which can undergo the foregoing ligand exchange, so that crosslinking between polymers does not occur during film formation, and the polarity changes with a lower amount of exposure as compared to a case where only another hypervalent iodine compound is used. That is, the resist composition of the invention, which has these characteristics, thus enables achievement of high sensitivity, high resolution, and low LWR.

As a resist composition for EUV lithography which is capable of forming a fine pattern, a metal resist based on a metal tin compound having a high ability to absorb EUV light like an iodine atom has been reported (for example, Patent Document 2). However, as described above, such metal resist compositions suffer from many problems including low solubility in solvents, poor shelf stability, and defectiveness due to post-etching residues which is unavoidable when a metal element is contained. On the other hand, the resist composition of the invention, which is free of a metal element, thus is more advantageous than a metal resist in terms of the level of defectiveness, and is well soluble in a solvent. Further, since the resist composition of the invention can be applied to both positive and negative types, it has a wide range of use applications. For example, in the contact hole forming step, the metal resist processed in negative development requires a reverse step after the formation of a pillar pattern, whereas the positive resist does not require such a step. Therefore, it can be said that the resist composition of the invention is more useful than the metal resist from the viewpoint of process simplicity.

JP-A 2015-180928 and JP-A 2018-095853 discloses a resist composition containing a hypervalent iodine compound as an additive and a resist composition in which a hypervalent iodine compound is incorporated in a polymer backbone of a base polymer. It is described in these patent documents that these resist compositions are successful only in improving line edge roughness. They refer nowhere to a possibility of photo-decomposition of the hypervalent iodine compound and an ability to function as a non-chemically amplified resist composition material. Further, according to the description regarding the compounding amount and specific examples, the hypervalent iodine compound is not a principal component in these resist compositions. Patent Document 3 discloses a positive resist composition comprising a hypervalent iodine compound, but does not indicate the hypervalent iodine compound having the formula (1) in the invention, and does not indicate that the use of the compound improves sensitivity and resolution. It is then believed that a non-chemically amplified resist composition material that exhibits extremely high sensitivity and excellent resolution and is very effective in micropatterning as in the invention is not conceivable from these patent documents. That is, the present invention provides a definitely novel resist composition and pattern forming process.

### [Pattern Forming Process]

When the resist composition is used in the fabrication of various integrated circuits, any well-known lithography techniques are applicable. For example, the invention provides a pattern forming process comprising the steps of applying the foregoing resist composition onto a substrate to form a resist film thereon, exposing the resist film to high-energy radiation, and optionally developing the exposed resist film in a developer.

First, the resist composition is applied onto a substrate for integrated circuit fabrication, an underlayer film laminated on a substrate (e.g., Si, SiO₂, SiN, SION, TiN, WSi, BPSG, SOG, or organic antireflective coating), a substrate for mask circuit fabrication, or an underlayer laminated on a substrate (e.g., Cr, CrO, CrON, MoSi₂, or SiO₂) by any suitable technique such as spin coating, roll coating, flow coating, dip coating, spray coating, or doctor coating. The coating is prebaked on a hot plate at a temperature of preferably 60 to 200° C. for 10 seconds to 30 minutes, more preferably at 80 to 180° C. for 30 seconds to 20 minutes to form a resist film having a thickness of 0.01 to 2 μm. The underlayer film means a film formed between the substrate and the resist film in a multi-layered resist forming process. The underlayer film is not limited, and may be a known underlayer film.

Next the resist film is exposed to high-energy radiation. The radiation is selected from among UV, deep UV, EB, EUV, X-ray, soft X-ray, excimer laser radiation, γ-ray, and synchrotron radiation. On use of UV, deep UV, EUV, X-ray, soft X-ray, excimer laser radiation, γ-ray, and synchrotron radiation as the high-energy radiation, the resist film is exposed thereto directly or through a mask having the desired pattern so as to reach a dose of preferably about 1 to 300 mJ/cm², more preferably about 10 to 200 mJ/cm². On use of EB as the high-energy radiation, imagewise writing is performed directly or through a mask having the desired pattern so as to reach a dose of preferably about 0.1 to 2,000 μC/cm², more preferably about 0.5 to 1,500 μC/cm². The resist composition is best suited in micropatterning using EB or EUV as the high-energy radiation.

If necessary, the resist film is post-exposure baked (PEB). Herein, the resist film is baked on a hotplate or in an oven preferably at 30 to 150° C. for 10 seconds to 30 minutes, and more preferably at 60 to 120° C. for 30 seconds to 20 minutes.

After the exposure or PEB, the resist film is developed in a developer to form a pattern, if necessary. The solvent used as the developer is preferably selected from alkali aqueous solutions such as aqueous tetramethylammonium hydroxide solutions; and organic solvents such as 2-octanone, 2-nonanone, 2-heptanone, 3-heptanone, 4-heptanone, 2-hexanone, 3-hexanone, diisobutyl ketone, methyl cyclohexanone, acetophenone, methylacetophenone, isopropyl alcohol, isoamyl alcohol, n-butanol, n-pentanol, cyclohexanol, propyl acetate, butyl acetate, isobutyl acetate, pentyl acetate, butenyl acetate, isopentyl acetate, cyclohexyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, 2-phenylethyl acetate, 2-propanol, 3-methoxybutanol, 3-methyl-3-methoxybutanol, 1-methoxy-2-propanol, 1-ethoxy-2-propanol, diacetone alcohol, and 4-methyl-2-pentanol. These developers may be used alone or in admixture of two or more.

At the end of development, the resist film is rinsed if necessary. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents. As the rinsing liquid, water may be used instead of the organic solvent.

Rinsing is effective for preventing the resist pattern from collapse or reducing defect formation. Rinsing is not essential. Rinsing is not essential. By omitting rinsing, the amount of the solvent used is saved.

### EXAMPLES

Synthesis Examples, Examples, and Comparative Examples of the invention are given below by way of illustration and not by way of limitation.

### [1] Synthesis of Hypervalent Iodine Compound

### [Synthesis Example 1-1] Synthesis of Hypervalent Iodine Compound I-1

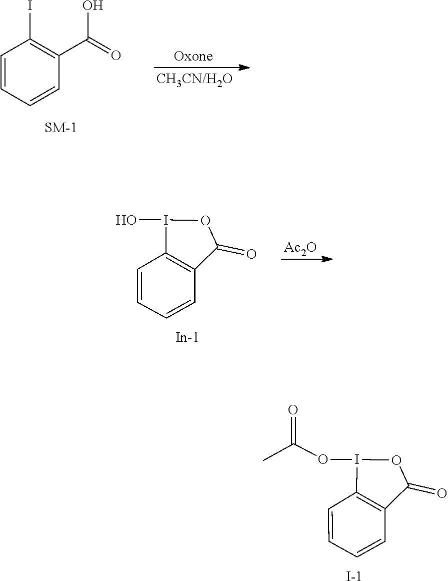

To a solution prepared by dissolving OXONE® (potassium peroxymonosulfate) (40 g) in water (40 g), a solution prepared by dissolving a reactant compound SM-1 (iodobenzoic acid) (13.6 g) in acetonitrile (40 g) was added dropwise at room temperature. After the dropwise addition, the mixture was stirred for 2 hours, and the precipitated solid was separated by filtration. The obtained solid was washed with water (40 mL), and then with acetone (40 mL), and dried under reduced pressure at 40° C. to obtain an intermediate In-1 as a solid (yield: 11.6 g, yield rate: 81%). Subsequently, the compound In-1 (8 g) and acetic anhydride (27 mL) were mixed, the mixture was stirred at 140° C. for 2 hours, the reaction liquid was returned to room temperature, and the precipitated solid was separated by filtration. The obtained solid was washed with diisopropyl ether, and dried under reduced pressure at 40° C. to obtain the titled hypervalent iodine compound I-1 as a solid (yield: 7.0 g, yield rate: 5%).

The results of nuclear magnetic resonance spectrometry (¹H-NMR/DMSO-d₆), single quadrupole mass spectrometry of the hypervalent iodine compound I-1 are shown below.

¹H-NMR (500 MHz, DMSO-d₆) δ 2.25 (s, 3H), 7.75 (m, 1H), 7.78 (m, 1H), 8.05 (m, 2H) ppm.

Single quadrupole mass spectrometry (ESI): POSITIVE M⁺H⁺ 307.0 (corresponding to C₉H₇IO₄)

### [Synthesis Example 1-2] Synthesis of Hypervalent Iodine Compound I-2

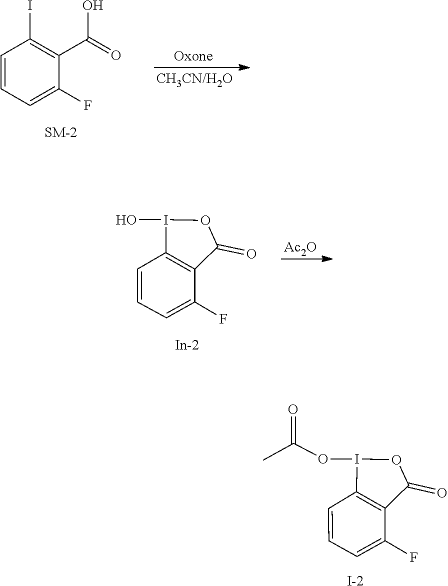

A hypervalent iodine compound I-2 was synthesized in the same manner as in Synthesis Example 1-1 except that a reactant compound SM-2 was used in place of the reactant compound SM-1 (yield rate: 86%).

The results of nuclear magnetic resonance spectrometry (¹H-NMR/DMSO-d₆), single quadrupole mass spectrometry of the hypervalent iodine compound I-2 are shown below.

¹H-NMR (500 MHz, DMSO-d₆) δ 2.21 (s, 3H), 7.61 (m, 1H), 7.72 (d, J=8.2 Hz, 1H), 7.95 (d, J=4.2 Hz, 1H) ppm.

Single quadrupole mass spectrometry (ESI): POSITIVE M⁺H⁺ 325.0 (corresponding to C₉H₆FIO₄)

### [Synthesis Example 1-3] Synthesis of Hypervalent Iodine Compound I-3

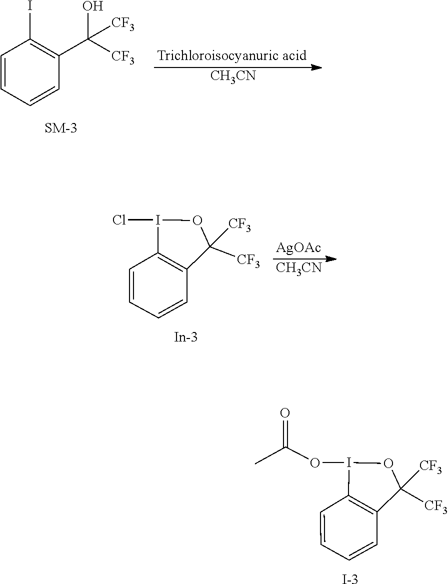

To a solution prepared by dissolving a reactant compound SM-3 (15 g) in acetonitrile (40 g), trichloroisocyanuric acid (3.4 g) was added. The mixture was stirred at room temperature for 30 minutes, and the precipitated solid was then separated by filtration to obtain an intermediate In-3 (amount 10.6 g, yield 65%).

Subsequently, IM-3 (8 g), silver acetate (3.3 g) and acetonitrile (100 g) were mixed, the mixture was then stirred at room temperature for 10 hours, and the precipitated solid was separated by filtration. The obtained solid was extracted with acetonitrile, and the extract was distilled off under reduced pressure to obtain the titled hypervalent iodine compound I-3 as a solid (yield: 8.5 g, yield rate: 100%).

The results of nuclear magnetic resonance spectrometry (¹H-NMR/CDCl₃), single quadrupole mass spectrometry of the hypervalent iodine compound I-3 are shown below.

¹H-NMR (500 MHz, CDCl₃) δ 2.18 (s, 3H), 7.61-7.79 (m, 3H), 7.93 (d, J=8.4 Hz, 1H) ppm.

Single quadrupole mass spectrometry (ESI): POSITIVE M⁺H⁺ 428.9 (corresponding to C₁₁H₈F₆IO₃)

### [2] Synthesis of Polymers

The following monomers were used for synthesis of polymers.

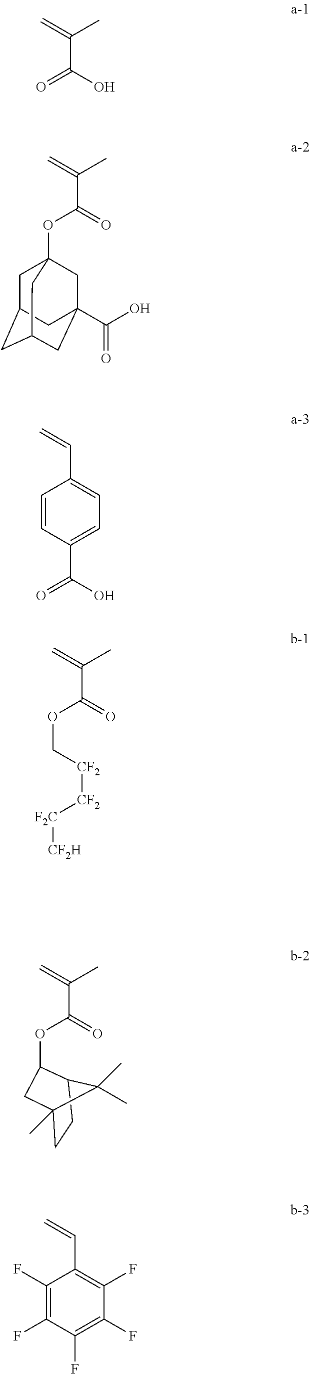

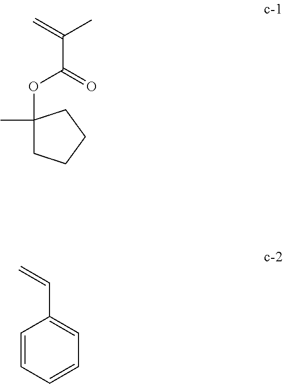

### [Synthesis Example 2-1] Synthesis of Polymer P-1

In a nitrogen atmosphere, a monomer a-1 (56 g), a monomer b-1 (105 g), V-601 (manufactured by FUJIFILM Wako Pure Chemical Corporation) (5.4 g), and MEK (180 g) were put into a flask to prepare a monomer-polymerization initiator solution. In another flask under a nitrogen atmosphere, 55 g of MEK was put and heated to 80° C. while stirred, and then the monomer-polymerization initiator solution was added dropwise over 4 hours. After completion of the dropwise addition, stir of the polymerization liquid was continued for 2 hours while the liquid temperature was maintained at 80° C., and then the polymerization liquid was cooled to room temperature. The obtained polymerization liquid was added dropwise to 4000 g of vigorously stirred hexane, and the precipitated polymer was separated by filtration. The obtained polymer was washed twice with hexane (1200 g), and then vacuum-dried at 50° C. for 20 hours to obtain a polymer P-1 in the form of a white powder (yield: 155 g, yield rate: 96%). The polymer P-1 had a value of Mw of 7700, and a value of Mw/Mn of 1.82. The value of Mw was measured by GPC versus polystyrene standards using THE as a solvent.

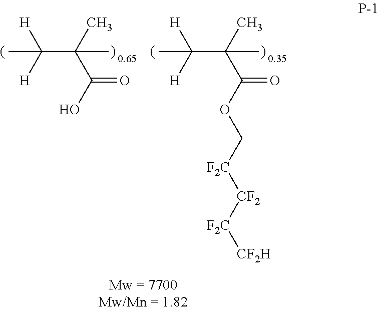

### [Synthesis Examples 2-2 to 2-10] Synthesis of Polymers P-2 to P-10

Polymers shown in Table 1 were synthesized in the same manner as in Synthesis Example 1 except that the kind and the compounding ratio of each monomer were changed.

TABLE 1

| | | | | Introduction | | Introduction | | |
|---|---|---|---|---|---|---|---|---|
| | | | | ratio | | ratio | | |
| | | Polymer | Unit 1 | (mol %) | Unit 2 | (mol %) | Mw | Mw/Mn |
| Synthesis | 1 | P-1 | a-1 | 65 | b-1 | 35 | 7,700 | 1.82 |
| Example | 2 | P-2 | a-1 | 50 | b-2 | 50 | 8,400 | 1.84 |
| | 3 | P-3 | a-1 | 60 | b-3 | 40 | 8,100 | 1.79 |
| | 4 | P-4 | a-2 | 65 | b-1 | 35 | 8,300 | 1.83 |
| | 5 | P-5 | a-2 | 50 | b-2 | 50 | 8,300 | 1.83 |
| | 6 | P-6 | a-2 | 60 | b-3 | 40 | 8,200 | 1.82 |
| | 7 | P-7 | a-3 | 65 | b-1 | 35 | 8,000 | 1.80 |
| | 8 | P-8 | a-3 | 50 | b-2 | 50 | 8,600 | 1.84 |
| | 9 | P-9 | a-3 | 60 | b-3 | 40 | 7,900 | 1.81 |
| | 10 | P-10 | c-1 | 60 | c-2 | 40 | 9,800 | 1.82 |

### [3] Preparation of Resist Composition

### Examples 1-1 to 1-16 and Comparative Examples 1-1 to 1-3

Resist compositions (R-01 to R-16) and a comparative resist composition (CR-01) were prepared by dissolving a hypervalent iodine compound, another hypervalent iodine compound and a polymer in a solvent containing 0.01 wt % of a surfactant (PF-636, manufactured by OMNOVA Solutions Inc.) in accordance with the recipe shown in Table 2, and filtering the solution through a Teflon® filter having a pore size of 0.2 μm. Separately, comparative resist compositions (CR-02 and CR-03) were prepared by dissolving a polymer, a photoacid generator, and a sensitivity modifier in a solvent containing 0.01 wt % of a surfactant (PF-636, manufactured by OMNOVA Solutions Inc.) in accordance with the recipe shown in Table 3, and filtering the solution through a Teflon® filter having a pore size of 0.2 μm.

TABLE 2

| | | | | Another | | | |
|---|---|---|---|---|---|---|---|
| | | | Hypervalent | hypervalent | | | |
| | | | iodine | iodine | | | |
| | | Resist | compound | compound | Polymer | Solvent | Solvent |
| | | composition | (pbw) | (pbw) | (pbw) | 1 (pbw) | 2 (pbw) |
| Example | 1-1 | R-01 | I-1 (10) | — | P-1 (8) | PGMEA (800) | AcOH (200) |
| | 1-2 | R-02 | I-2 (10) | — | P-1 (8) | PGMEA (800) | AcOH (200) |
| | 1-3 | R-03 | I-3 (14) | — | P-1 (8) | PGMEA (800) | AcOH (200) |
| | 1-4 | R-04 | I-1 (10) | — | P-2 (10) | PGMEA (800) | AcOH (200) |
| | 1-5 | R-05 | I-1 (10) | — | P-3 (7) | PGMEA (800) | AcOH (200) |
| | 1-6 | R-06 | I-1 (10) | — | P-4 (11) | PGMEA (800) | AcOH (200) |
| | 1-7 | R-07 | I-1 (10) | — | P-5 (16) | PGMEA (800) | AcOH (200) |
| | 1-8 | R-08 | I-1 (10) | — | P-6 (13) | PGMEA (800) | AcOH (200) |
| | 1-9 | R-09 | I-1 (10) | — | P-7 (10) | PGMEA (800) | AcOH (200) |
| | 1-10 | R-10 | I-1 (10) | — | P-8 (12) | PGMEA (800) | AcOH (200) |
| | 1-11 | R-11 | I-1 (10) | — | P-9 (9) | PGMEA (800) | AcOH (200) |
| | 1-12 | R-12 | I-1 (10) | — | P-1 (8) | HBM (800) | AcOH (200) |
| | 1-13 | R-13 | I-1 (10) | — | P-1 (8) | PGMEA (800) | PA (200) |
| | 1-14 | R-14 | I-1 (5) | — | P-1 (8) | PGMEA (800) | AcOH (200) |
| | 1-15 | R-15 | I-1 (5) | O-1 (2.5) | P-1 (8) | PGMEA (800) | AcOH (200) |
| | 1-16 | R-16 | I-1 (5) | — | P-1 (8) | PGMEA (800) | AcOH (200) |
| | | | I-2 (5) | | | | |
| Comparative | 1-1 | CR-01 | — | O-1 (10) | P-1 (15) | PGMEA (800) | AcOH (200) |
| Example | | | | | | | |

TABLE 3

| | | | | Photoacid | Sensitivity | | |
|---|---|---|---|---|---|---|---|
| | | Resist | Polymer | generator | modifier | Solvent 1 | Solvent 2 |
| | | composition | (pbw) | (pbw) | (pbw) | (pbw) | (pbw) |
| Comparative | 1-2 | CR-02 | P-10 (80) | PAG-1 (19) | Q-1 (6) | PGMEA (1890) | GBL (210) |
| Example | 1-3 | CR-03 | P-10 (80) | PAG-1 (19) | I-1 (5) | PGMEA (1890) | GBL (210) |

In Tables 2 and 3, the another hypervalent iodine compound O-1, the photoacid generator PAG-1, the sensitivity modifier Q-1, and the solvent are identified below.

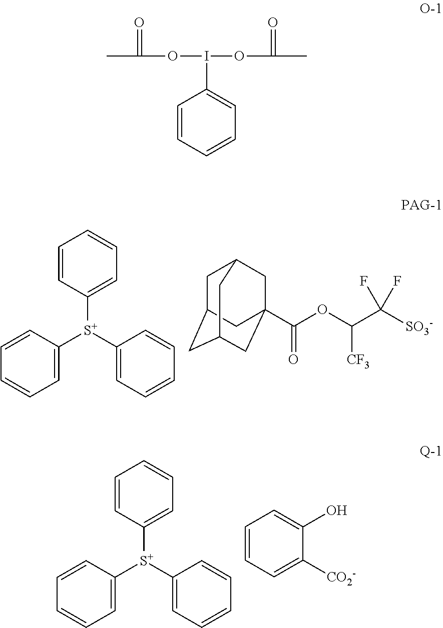

### Solvent:

PGMEA (propylene glycol monomethyl ether acetate)
AcOH (acetic acid)
HBM (methyl 2-hydroxyisobutyrate)
PA (propionic acid)
GBL (γ-butyrolactone)

### [4] EUV Lithography Test (Line-and-Space Pattern, Positive-Tone Development)

### Examples 2-1 to 2-16 and Comparative Examples 2-1 to 2-3

Each of the resist compositions (R-01 to R-16, CR-01 to CR-03) was spin coated on a silicon substrate having a 20-nm coating of silicon-containing spin-on hard mask SHB-A940 (Shin-Etsu Chemical Co., Ltd., silicon content 43 wt %) and prebaked (PAB) on a hotplate at the temperature shown in Table 4 for 60 seconds to form a resist film of 40 nm thick. Using an EUV scanner NXE3400 (ASML, NA 0.33, σ 0.9, 90° dipole illumination), the resist film was exposed to EUV through a mask bearing a 36-nm 1:1 line-and-space (LS) pattern. The resist film was baked (PEB) on a hotplate at the temperature shown in Table 4 for 60 seconds and developed in the developer shown in Table 4 for 30 seconds to form an LS pattern having a space width of 18 nm and a pitch of 36 nm.

The obtained resist pattern was evaluated as follows. Table 4 shows the results.

### [Evaluation of Sensitivity]

The LS pattern was observed under CD-SEM (CG-6300, Hitachi High-Tech Corporation), and the optimum dose (Eop, mJ/cm²) which provided an LS pattern with a space width of 18 nm and a pitch of 36 nm was determined and reported as sensitivity.

### [Evaluation of LWR]

An LS pattern was formed by exposure in the optimum dose (Eop). The space width was measured under CD-SEM (CG-6300, Hitachi High-Tech Corporation) at longitudinally spaced apart 10 points, from which a 3-fold value (36) of the standard deviation (o) was determined and reported as LWR. A smaller value indicates a pattern having a lower roughness and more uniform space width.

### [Evaluation of Maximum Resolution]

An LS pattern was formed while increasing the exposure dose little by little from the optimum dose (Eop). The line width (nm) which could be resolved was determined under CD-SEM (CG-6300, Hitachi High-Tech Corporation) and reported as maximum resolution (nm). A smaller value indicates a pattern having a better maximum resolution and smaller

TABLE 4

| | | Resist | PAB/PEB | | Eop | LWR | Maximum resolution |
|---|---|---|---|---|---|---|---|
| | | composition | (° C.) | Developer | (mJ/cm²) | (nm) | (nm) |
| Example | 2-1 | R-01 | 130/90 | nBA | 29 | 3.0 | 10 |
| | 2-2 | R-02 | 130/90 | nBA | 27 | 2.5 | 9 |
| | 2-3 | R-03 | 130/90 | nBA | 30 | 2.9 | 10 |
| | 2-4 | R-04 | 130/90 | nBA | 32 | 2.8 | 9 |
| | 2-5 | R-05 | 130/90 | nBA | 34 | 2.5 | 9 |
| | 2-6 | R-06 | 130/90 | nBA | 35 | 2.7 | 11 |
| | 2-7 | R-07 | 130/90 | nBA | 34 | 2.8 | 12 |
| | 2-8 | R-08 | 130/90 | nBA | 35 | 2.6 | 12 |
| | 2-9 | R-09 | 130/90 | nBA | 30 | 2.5 | 12 |
| | 2-10 | R-10 | 130/90 | nBA | 32 | 2.7 | 10 |
| | 2-11 | R-11 | 130/90 | nBA | 32 | 2.2 | 12 |
| | 2-12 | R-12 | 130/90 | nBA | 29 | 2.2 | 9 |
| | 2-13 | R-13 | 130/90 | CHA | 26 | 2.4 | 9 |
| | 2-14 | R-14 | 130/90 | nBA | 24 | 2.2 | 8 |
| | 2-15 | R-15 | 130/90 | nBA | 35 | 2.3 | 9 |
| | 2-16 | R-16 | 130/90 | nBA | 28 | 2.7 | 9 |
| Comparative | 2-1 | CR-01 | 130/90 | nBA | 39 | 3.6 | 14 |
| Example | 2-2 | CR-02 | 105/90 | TMAH | 80 | 4.5 | 18 |
| | 2-3 | CR-03 | 105/90 | TMAH | 85 | 4.8 | 18 |

### Developer:

nBA (butyl acetate)
CHA (cyclohexyl acetate)
TMAH (2.38 wt % aqueous tetramethylammonium hydroxide solution)

### [5] EUV Lithography Test (Line-and-Space Pattern, Negative-Tone Development)

### Examples 3-1 to 3-16 and Comparative Examples 3-1 to 3-3

Each of the resist compositions (R-1 to R-16, CR-01 to CR-03) was spin coated on a silicon substrate having a 20-nm coating of silicon-containing spin-on hard mask SHB-A940 (Shin-Etsu Chemical Co., Ltd., silicon content 43 wt %) and prebaked (PAB) on a hotplate at the temperature shown in Table 5 for 60 seconds to form a resist film of 40 nm thick. Using an EUV scanner NXE3400 (ASML, NA 0.33, σ 0.9, 90° dipole illumination), the resist film was exposed to EUV through a mask bearing a 36-nm 1:1 line-and-space (LS) pattern. The resist film was baked (PEB) on a hotplate at the temperature shown in Table 5 for 60 seconds and developed in the developer shown in Table 5 for 30 seconds to form an LS pattern having a space width of 18 nm and a pitch of 36 nm.

The obtained resist pattern was evaluated as follows. Table 4 shows the results.

### [Evaluation of Sensitivity]

The LS pattern was observed under CD-SEM (CG-6300, Hitachi High-Tech Corporation), and the optimum dose (Eop, mJ/cm²) which provided an LS pattern with a space width of 18 nm and a pitch of 36 nm was determined and reported as sensitivity.

### [Evaluation of LWR]

An LS pattern was formed by exposure in the optimum dose (Eop). The space width was measured under CD-SEM (CG-6300, Hitachi High-Tech Corporation) at longitudinally spaced apart 10 points, from which a 3-fold value (36) of the standard deviation (o) was determined and reported as LWR. A smaller value indicates a pattern having a lower roughness and more uniform space width.

### [Evaluation of Maximum Resolution]

An LS pattern was formed while increasing the exposure dose little by little from the optimum dose (Eop). The line width (nm) which could be resolved was determined under CD-SEM (CG-6300, Hitachi High-Tech Corporation) and reported as maximum resolution (nm). A smaller value indicates a pattern having a better maximum resolution and smaller

TABLE 5

| | | Resist | PAB/PEB | | Eop | LWR | Maximum resolution |
|---|---|---|---|---|---|---|---|
| | | composition | (° C.) | Developer | (mJ/cm²) | (nm) | (nm) |
| Example | 3-1 | R-01 | 130/90 | TMAH | 30 | 3.5 | 11 |
| | 3-2 | R-02 | 130/90 | TMAH | 28 | 2.9 | 10 |
| | 3-3 | R-03 | 130/90 | TMAH | 31 | 3.4 | 12 |
| | 3-4 | R-04 | 130/90 | TMAH | 33 | 3.3 | 10 |
| | 3-5 | R-05 | 130/90 | TMAH | 35 | 2.9 | 10 |
| | 3-6 | R-06 | 130/90 | TMAH | 36 | 3.2 | 13 |
| | 3-7 | R-07 | 130/90 | TMAH | 35 | 2.3 | 13 |
| | 3-8 | R-08 | 130/90 | TMAH | 36 | 3.1 | 13 |
| | 3-9 | R-09 | 130/90 | TMAH | 31 | 3.0 | 14 |
| | 3-10 | R-10 | 130/90 | TMAH | 33 | 3.2 | 11 |
| | 3-11 | R-11 | 130/90 | TMAH | 33 | 2.7 | 13 |
| | 3-12 | R-12 | 130/90 | TMAH | 30 | 2.7 | 10 |
| | 3-13 | R-13 | 130/90 | TMAH | 27 | 2.8 | 10 |
| | 3-14 | R-14 | 130/90 | TMAH | 25 | 2.7 | 9 |
| | 3-15 | R-15 | 130/90 | TMAH | 36 | 2.8 | 10 |
| | 3-16 | R-16 | 130/90 | TMAH | 29 | 3.2 | 10 |
| Comparative | 3-1 | CR-01 | 130/90 | TMAH | 40 | 4.1 | 16 |
| Example | 3-2 | CR-02 | 105/90 | nBA | 83 | 4.6 | 18 |
| | 3-3 | CR-03 | 105/90 | nBA | 86 | 4.9 | 18 |

It is evident from Tables 4 and 5 that the resist compositions within the scope of the invention form, whether in positive-tone or negative-tone development, line-and-space patterns having satisfactory sensitivity, LWR, and resolution when processed by EUV lithography.

### [6] EUV Lithography Test (Contact Hole Pattern)

### Examples 4-1 to 4-16 and Comparative Examples 4-1 to 4-3

Each of the resist compositions (R-01 to R-16, CR-01 to CR-03) was spin coated on a silicon substrate having a 20-nm coating of silicon-containing spin-on hard mask SHB-A940 (Shin-Etsu Chemical Co., Ltd., silicon content 43 wt %) and prebaked (PAB) on a hotplate at the temperature shown in Table 6 for 60 seconds to form a resist film of 50 nm thick. Using an EUV scanner NXE3400 (ASML, NA 0.33, σ 0.9/0.6, quadrupole illumination), the resist film was exposed to EUV through a mask bearing a hole pattern having a pitch of 64 nm (on-wafer size) and +20% bias. The resist film was baked (PEB) on a hotplate at the temperature shown in Table 6 for 60 seconds and developed in developer shown in Table 6 for 30 seconds to obtain a hole pattern with a size of 32 nm.

The obtained resist pattern was evaluated as follows. Table 6 shows the results.

### [Evaluation of Sensitivity]

The contact hole pattern was observed under CD-SEM (CG-6300, Hitachi High-Tech Corporation), and the optimum dose (Eop, mJ/cm²) which provided a hole pattern with a size of 32 nm was determined.

### [Evaluation of CD Uniformity (CDU)]

The size of 50 holes which were printed at Eop was measured, from which a 3-fold value (3σ) of the standard deviation (σ) was computed and reported as CDU. A smaller value of CDU indicates a hole pattern with more uniform hole diameter.

### [Evaluation of Maximum Resolution]

A hole pattern was formed while decreasing the exposure dose little by little from the optimum dose (Eop). The hole diameter (nm) which could be resolved was determined under CD-SEM (CG-6300, Hitachi High-Tech Corporation) and reported as maximum resolution (nm). A smaller value indicates a pattern having a better maximum resolution and smaller hole diameter.

TABLE 6

| | | Resist | PAB/PEB | | Eop | CDU | Maximum resolution |
|---|---|---|---|---|---|---|---|
| | | composition | (° C.) | Developer | (mJ/cm²) | (nm) | (nm) |
| Example | 4-1 | R-01 | 130/90 | nBA | 19 | 2.0 | 20 |
| | 4-2 | R-02 | 130/90 | nBA | 17 | 2.3 | 20 |
| | 4-3 | R-03 | 130/90 | nBA | 20 | 2.2 | 21 |
| | 4-4 | R-04 | 130/90 | nBA | 22 | 1.8 | 20 |
| | 4-5 | R-05 | 130/90 | nBA | 21 | 2.0 | 22 |
| | 4-6 | R-06 | 130/90 | nBA | 20 | 1.9 | 20 |
| | 4-7 | R-07 | 130/90 | nBA | 21 | 1.8 | 23 |
| | 4-8 | R-08 | 130/90 | nBA | 20 | 1.7 | 23 |
| | 4-9 | R-09 | 130/90 | nBA | 19 | 1.7 | 23 |
| | 4-10 | R-10 | 130/90 | nBA | 21 | 1.8 | 22 |
| | 4-11 | R-11 | 130/90 | nBA | 21 | 1.7 | 24 |
| | 4-12 | R-12 | 130/90 | nBA | 18 | 1.7 | 19 |
| | 4-13 | R-13 | 130/90 | CHA | 18 | 2.0 | 19 |
| | 4-14 | R-14 | 130/90 | nBA | 16 | 1.6 | 18 |
| | 4-15 | R-15 | 130/90 | nBA | 21 | 1.9 | 19 |
| | 4-16 | R-16 | 130/90 | nBA | 18 | 2.2 | 20 |
| Comparative | 4-1 | CR-01 | 130/90 | nBA | 24 | 2.7 | 26 |
| Example | 4-2 | CR-02 | 105/90 | TMAH | 42 | 3.8 | 32 |
| | 4-3 | CR-03 | 105/90 | TMAH | 40 | 4.0 | 32 |

It is evident from Table 6 that the resist compositions within the scope of the invention form contact hole patterns having satisfactory sensitivity, CDU, and resolution when processed by the EUV lithography.

Japanese Patent Application No. 2024-059980 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. A resist composition comprising a hypervalent iodine compound having the formula (1), a carboxy group-containing polymer, and a solvent: 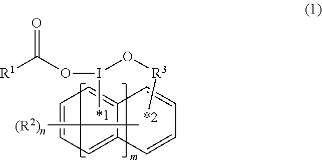
wherein m is 0 or 1, n is an integer of 0 to 4 when m is 0, and an integer of 0 to 6 when m is 1,
R¹ is halogen, or a C₁-C₁₀ hydrocarbyl group which may contain a heteroatom,
R² is halogen, or a C₁-C₄₀ hydrocarbyl group which may contain a heteroatom, R² may be the same or different when n is 2 or more, a plurality of R² may bond together to form a ring with the aromatic ring carbon atoms to which they are attached,
R³ is a carbonyl group, or C₁-C₁₀ hydrocarbylene group which may contain a heteroatom, and
1 and *2 each designate a valence bond to a carbon atom in an aromatic ring, provided that *1 and *2 bond to adjacent carbon atoms in the aromatic ring.

2. The resist composition of claim 1, wherein the carboxy group-containing polymer contains recurring units having the formula (2): 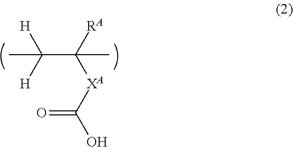
wherein R^{A} is a hydrogen atom, halogen, methyl group, or trifluoromethyl group,
X^{A} is a single bond, phenylene group, naphthylene group, or *—C(═O)—O—X^{A1}—,
wherein X^{A1} is a C₁-C₁₀ saturated hydrocarbylene group, phenylene group, or naphthylene group, the saturated hydrocarbylene group may contain at least one selected from a hydroxy group, an ether bond, an ester bond and a lactone ring, and * designates a valence bond to a carbon atom in a main chain.

3. The resist composition of claim 1, further comprising a hypervalent iodine compound having the formula (3): 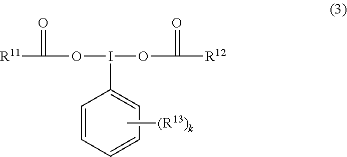
wherein k is an integer of 0 to 5,
R¹¹ and R¹² are each independently halogen or a C₁-C₁₀ hydrocarbyl group which may contain a heteroatom, R¹¹ and R¹² may bond together to form a ring with the carbon atoms to which they are attached, and atoms between the carbon atoms,
R¹³ is halogen, or a C₁-C₄₀ hydrocarbyl group which may contain a heteroatom, R¹³ may be the same or different when k is 2, 3, 4 or 5, and a plurality of R¹³ may bond together to form a ring with the aromatic ring carbon atoms to which they are attached.

4. A laminate comprising a substrate, and a resist film obtained from the resist composition of claim 1.

5. The laminate of claim 4 comprising a resist underlayer film between the substrate and the resist film.

6. A pattern forming process comprising the steps of applying the resist composition of claim 1 onto a substrate, or an underlayer film laminated on a substrate to form a resist film thereon, exposing the resist film to a high-energy radiation, and developing the exposed resist film in a developer.

7. The pattern forming process of claim 6, wherein the high-energy radiation is an electron beam or an extreme ultraviolet radiation.

8. The pattern forming process of claim 6, wherein the developer dissolves exposed regions and does not dissolve unexposed regions.

9. The pattern forming process of claim 6, wherein the developer dissolves unexposed regions and does not dissolve exposed regions.

10. A laminate comprising a substrate, and a resist film on the substrate, wherein the resist film contains a polymer containing a recurring unit having the formula (4): 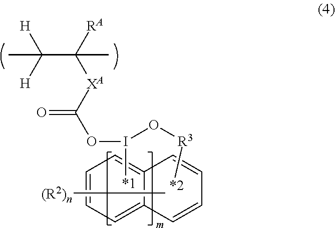
wherein m is 0 or 1, n is an integer of 0 to 4 when m is 0, and an integer of 0 to 6 when m is 1,
R² is halogen, or a C₁-C₄₀ hydrocarbyl group which may contain a heteroatom, R² may be the same or different when n is 2 or more, a plurality of R² may bond together to form a ring with the aromatic ring carbon atoms to which they are attached,
R³ is a carbonyl group, or C₁-C₁₀ hydrocarbylene group which may contain a heteroatom,
*1 and *2 each designate a valence bond to a carbon atom in an aromatic ring, provided that *1 and *2 bond to adjacent carbon atoms in the aromatic ring,
R^{A} is a hydrogen atom, halogen, methyl group or trifluoromethyl group,
X^{A} is a single bond, phenylene group, naphthylene group, or *—C(═O)—O—X^{A1}—, wherein X^{A1} is a C₁-C₁₀ saturated hydrocarbylene group, phenylene group, or naphthylene group, the saturated hydrocarbylene group may contain at least one selected from a hydroxy group, an ether bond, an ester bond and a lactone ring, and * designates a valence bond to a carbon atom in a main chain.

11. The laminate of claim 10 comprising a resist underlayer film between the substrate and the resist film.

12. A pattern forming process comprising the steps of exposing the resist film of the laminate of the claim 10 to high-energy radiation, and developing the exposed resist film in a developer.

13. The pattern forming process of claim 12, wherein the high-energy radiation is an electron beam or an extreme ultraviolet radiation.

14. The pattern forming process of claim 12, wherein the developer dissolves exposed regions and does not dissolve unexposed regions.

15. The pattern forming process of claim 12, wherein the developer dissolves unexposed regions and does not dissolve exposed regions.
